SEQUENCE LISTING (58 sequences)

SEQ ID NO: 1 - protein, 20 aa, Artificial Sequence
  Description of Artificial Sequencepeptide 35, peptide 88, peptide 35/88
RLRRICSGILLIRRILGIFV

SEQ ID NO: 2 - protein, 4 aa, Artificial Sequence
  Description of Artificial SequenceC-terminus vector-derived sequence
RPVR

SEQ ID NO: 3 - protein, 5 aa, Artificial Sequence
  Description of Artificial SequenceC-terminus vector-derived sequence
RPVRP

SEQ ID NO: 4 - protein, 17 aa, Artificial Sequence
  Description of Artificial Sequencepeptide 38
TSGLLKLVQAKRKCCIS

SEQ ID NO: 5 - protein, 20 aa, Artificial Sequence
  Description of Artificial Sequencepeptide 40
RWDPTRLLRFRFLRMLVRRS

SEQ ID NO: 6 - protein, 20 aa, Artificial Sequence
  Description of Artificial Sequencepeptide 41
GRGCIFRWRRGLRGMMRLFK

SEQ ID NO: 7 - protein, 7 aa, Artificial Sequence
  Description of Artificial Sequencelysine residues fused to N-terminus, K7, lys7
KKKKKKK

SEQ ID NO: 8 - DNA, 78 bp, Artificial Sequence
  Description of Artificial Sequencenucleotide sequence encoding peptide 35/88
cggctccggagaatatgtagcggcattctgctcatccgtaggatattgggcattttcgttaggcccgtgaggccctaa

SEQ ID NO: 9 - DNA, 53 bp, Artificial Sequence
  Description of Artificial Sequencenucleotide sequence encoding peptide 38
actagtgggttgctgaagctggtgcaggctaagcgtaagtgttgtattagtta

SEQ ID NO: 10 - DNA, 78 bp, Artificial Sequence
  Description of Artificial Sequencenucleotide sequence encoding peptide 40
cgttgggatccgacgcgattgctgcgatttcggttcctccggatgctagtgaggcggagtaggcccgtgaggccctaa

SEQ ID NO: 11 - DNA, 63 bp, Artificial Sequence
  Description of Artificial Sequencenucleotide sequence encoding peptide 41
ggaaggggatgtatctttcgatggaggagaggcctgcggggaatgatgagactatttaagtag

SEQ ID NO: 12 - protein, 8 aa, Artificial Sequence
  MOD_RES (1)..(3): Xaa = large hydrophobic amino acid selected from the group Leu, Ile, Phe, Met, Tyr or Trp, wherein at least one is Leu or Ile, Xaa at position 3 may be present or absent
XXXXXXXX

SEQ ID NO: 13 - protein, 6 aa, Artificial Sequence
  Description of Artificial Sequencelinker
EEAAKA

SEQ ID NO: 14 - protein, 37 aa, Artificial Sequence
  Description of Artificial Sequencebiotinylated peptide 40 fused to C-terminus of GFP
XMDELYKEEAAKARWDPTRLLRFRFLRMLVRRSRPVR

SEQ ID NO: 15 - protein, 37 aa, Artificial Sequence
  Description of Artificial Sequenceinactive biotinylated alanine mutant of peptide 40
XMDELYKEEAAKARWDPTRALRARFARALVRRSRPVR

SEQ ID NO: 16 - protein, 33 aa, Artificial Sequence
  Description of Artificial Sequencepeptide 41 fused to C-terminus of GFP
GMDELYKEEAAKAGRGCIFRWRRGLRGMMRLFK

SEQ ID NO: 17 - DNA, 54 bp, Artificial Sequence
  Description of Artificial Sequencenucleotide sequence of peptide 38 with in-frame stop codon
actagtgggttgctgaagctggtgcaggctaagcgtaagtgttgtattagttag

SEQ ID NO: 18 - protein, 25 aa, Artificial Sequence
  Description of Artificial Sequencepeptide #40
RWDPTRLLRFRFLRMLVRRSRPVRP

SEQ ID NO: 19 - protein, 25 aa, Artificial Sequence
  Description of Artificial Sequencepeptide #88
RLRRICSGILLIRRILGIFVRPVRP

SEQ ID NO: 20 - protein, 40 aa, Artificial Sequence
  Description of Artificial Sequencevariant of synthetic peptide #40 with N-terminus seven Lys and linker, K7_40
KKKKKKKGGEEAAKARWDPTRLLRFRFLRMLVRRSRPVRP

SEQ ID NO: 21 - protein, 40 aa, Artificial Sequence
  Description of Artificial Sequencevariant of synthetic peptide #40 with N-terminus seven Lys and linker and alanine mutated residues, K7_40 M
KKKKKKKGGEEAAKARWDPTRALRARFARALVRRSRPVRP

SEQ ID NO: 22 - protein, 35 aa, Artificial Sequence
  Description of Artificial Sequencevariant of synthetic peptide #41 with N-terminus seven Lys and linker, K7_41
KKKKKKKGGEEAAKAGRGCIFRWRRGLRGMMRLFK

SEQ ID NO: 23 - protein, 35 aa, Artificial Sequence
  Description of Artificial Sequencevariant of synthetic peptide #41 with N-terminus seven Lys and linker and alanine mutated residues, K7_41 M
KKKKKKKGGEEAAKAGRGCIFRARRGARGMARAFK

SEQ ID NO: 24 - protein, 5 aa, Artificial Sequence
  Description of Artificial Sequence5 arginine residues
RRRRR

SEQ ID NO: 25 - protein, 8 aa, Artificial Sequence
  Description of Artificial Sequencelinker
GGEEAAKA

SEQ ID NO: 26 - protein, 13 aa, Artificial Sequence
  Description of Artificial SequenceC-terminus of GFP and linker fused to biotinylated peptide 40, 41 and 35
GMDELYKEEAAKA

SEQ ID NO: 27 - protein, 200 aa, Artificial Sequence
  Description of Artificial Sequencepoly Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 28 - protein, 24 aa, Artificial Sequence
  Description of Artificial Sequence proliferating cell nuclear antigen (PCNA)-binding C-terminal peptide of tumor suppressor p21 (p21C)
KRRQTSMTDFYHSKRRLIFSKRKP

SEQ ID NO: 29 - protein, 25 aa, Artificial Sequence
  Description of Artificial Sequencepeptide #40 alanine mutant (M15A)
RWDPTRLLRFRFLRALVRRSRPVRP

SEQ ID NO: 30 - protein, 25 aa, Artificial Sequence
  Description of Artificial Sequencepeptide #40 alanine mutant (L13A/M15A)
RWDPTRLLRFRFARALVRRSRPVRP

SEQ ID NO: 31 - protein, 25 aa, Artificial Sequence
  Description of Artificial Sequencepeptide #40 alanine mutant (F10A/L13A/M15A)
RWDPTRLLRARFARALVRRSRPVRP

SEQ ID NO: 32 - protein, 20 aa, Artificial Sequence
  Description of Artificial Sequencepeptide #41 alanine mutant (L18A)
GRGCIFRWRRGLRGMMRAFK

SEQ ID NO: 33 - protein, 20 aa, Artificial Sequence
  Description of Artificial Sequencepeptide #41 alanine mutant (M16A/L18A)
GRGCIFRWRRGLRGMARAFK

SEQ ID NO: 34 - protein, 20 aa, Artificial Sequence
  Description of Artificial Sequencepeptide #41 alanine mutant (L12A/M16A/L18A)
GRGCIFRWRRGARGMARAFK

SEQ ID NO: 35 - protein, 25 aa, Artificial Sequence
  Description of Artificial Sequencepeptide #88 alanine mutant (I18A)
RLRRICSGILLIRRILGAFVRPVRP

SEQ ID NO: 36 - protein, 25 aa, Artificial Sequence
  Description of Artificial Sequencepeptide #88 alanine mutant (L16A/I18A)
RLRRICSGILLIRRIAGAFVRPVRP

SEQ ID NO: 37 - protein, 25 aa, Artificial Sequence
  Description of Artificial Sequencepeptide #88 alanine mutant (I12A/L16A/I18A)
RLRRICSGILLARRIAGAFVRPVRP

SEQ ID NO: 38 - protein, 10 aa, Artificial Sequence
  Description of Artificial Sequenceleucine-rich motif of HIV-1 Rev (amino acid position 75-84)
LPPLERLTLD

SEQ ID NO: 39 - protein, 11 aa, Artificial Sequence
  Description of Artificial Sequenceleucine-rich motif of Mitogen-activated protein kinase kinase 1, MAPKK (amino acid position 33-43)
LQKKLEELELD

SEQ ID NO: 40 - protein, 12 aa, Artificial Sequence
  Description of Artificial Sequenceleucine-rich motif of HTLV-1 Rex (amino acid position 82-93)
LSAQLYSSLSLD

SEQ ID NO: 41 - protein, 12 aa, Artificial Sequence
  Description of Artificial Sequenceleucine-rich motif of Human homologue of mouse double minute 2, Hdm-2 (amino acid position 190-200)
ISLSFDESLALC

SEQ ID NO: 42 - protein, 11 aa, Artificial Sequence
  Description of Artificial Sequenceleucine-rich motif of Protein kinase inhibitor, PKI (amino acid position 38-48)
LALKLAGLDIN

SEQ ID NO: 43 - protein, 17 aa, Artificial Sequence
  Description of Artificial SequenceC-terminal 17 residues of cell division cycle 42 isoform 2, CDC42C (amino acid position 183-191)
AALEPPETQPKRKCCIF

SEQ ID NO: 44 - protein, 9 aa, Artificial Sequence
  Description of Artificial Sequencepeptide #38Ndelta(1-8)
QAKRKCCIS

SEQ ID NO: 45 - protein, 4 aa, Artificial Sequence
  Description of Artificial Sequencepeptide #38Ndelta(1-13)
CCIS

SEQ ID NO: 46 - protein, 17 aa, Artificial Sequence
  Description of Artificial Sequencepeptide #38(T1A)
ASGLLKLVQAKRKCCIS

SEQ ID NO: 47 - protein, 17 aa, Artificial Sequence
  Description of Artificial Sequencepeptide #38(S2A)
TAGLLKLVQAKRKCCIS

SEQ ID NO: 48 - protein, 17 aa, Artificial Sequence
  Description of Artificial Sequencepeptide #38(G3A)
TSALLKLVQAKRKCCIS

SEQ ID NO: 49 - protein, 17 aa, Artificial Sequence
  Description of Artificial Sequencepeptide #38(L4A)
TSGALKLVQAKRKCCIS

SEQ ID NO: 50 - protein, 17 aa, Artificial Sequence
  Description of Artificial Sequencepeptide #38(L5A)
TSGLAKLVQAKRKCCIS

SEQ ID NO: 51 - protein, 17 aa, Artificial Sequence
  Description of Artificial Sequencepeptide #38(K6A)
TSGLLALVQAKRKCCIS

SEQ ID NO: 52 - protein, 17 aa, Artificial Sequence
  Description of Artificial Sequencepeptide #38(L7A)
TSGLLKAVQAKRKCCIS

SEQ ID NO: 53 - protein, 17 aa, Artificial Sequence
  Description of Artificial Sequencepeptide #38(V8A)
TSGLLKLAQAKRKCCIS

SEQ ID NO: 54 - protein, 17 aa, Artificial Sequence
  Description of Artificial Sequencepeptide #38(C14A)
TSGLLKLVQAKRKACIS

SEQ ID NO: 55 - protein, 24 aa, Artificial Sequence
  Description of Artificial Sequencepeptide 40
RWDPTRLLRFRFLRMLVRRSRPVR

SEQ ID NO: 56 - protein, 12 aa, Artificial Sequence
  Description of Artificial SequenceHuman homologue of mouse double minute 2, HDM-2
LSLSFDESLALC

SEQ ID NO: 57 - protein, 13 aa, Artificial Sequence
  Description of Artificial SequenceN-terminal sequence of peptides 35, 40 and 41 synthesized with C-terminus of GFP and spacer residues
XMDELYKEEAAKA

SEQ ID NO: 58 - protein, 6 aa, Artificial Sequence
  Description of Artificial SequenceC-terminus of GFP
MDELYK

(19) United States
(12) Patent Application Publication
(10) Pub. No.: US 2006/0216299 A1
(43) Pub. Date: Sep. 28, 2006

(54) Cell cycle targets and peptides

(75) Inventors: Yasumichi Hitoshi, Brisbane, CA (US); David Anderson, San Bruno, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Rigel Pharmaceuticals, Inc., South San Francisco, CA (US)

(21) Appl. No.: 10/531,492

(22) PCT Filed: Oct. 30, 2003
(86) PCT No.: PCT/US03/34669

§ 371 Date: Apr. 15, 2005

Related U.S. Application Data
(60) Provisional application No. 60/422,912, filed on Oct. 30, 2002.
(60) Provisional application No. 60/460,845, filed on Apr. 4, 2003.

(51) Int. Cl. A61K 48/00; C12Q 1/68; G01N 33/574; C07K 14/82; C07K 7/64; A61K 39/395

(52) U.S. Cl. 424/155.1; 435/6; 435/7.23; 514/44; 514/9; 530/317; 530/350

(57) ABSTRACT

The present invention relates to regulation of cellular proliferation. More particularly, the present invention binding partners of peptides that block the cell cycle. These binding partners are targets for drug discovery, e.g., for small molecule cancer therapeutics.

### CROSS-REFERENCES TO RELATED APPLICATIONS

This application claims the benefit of U.S. provisional application 60/422,912, filed Oct. 30, 2002; and claims the benefit of U.S. provisional application 60/460,845, filed Apr. 4, 2003; both of which are herein incorporated by reference in their entirety for all purposes.

### STATEMENT AS TO RIGHTS TO INVENTIONS MADE UNDER FEDERALLY SPONSORED RESEARCH AND DEVELOPMENT

Not applicable.

### FIELD OF THE INVENTION

The present invention relates to regulation of cellular proliferation. More particularly, the present invention relates to binding partners of peptides that block the cell cycle. These binding partners and the corresponding peptides are targets for drug discovery, e.g., for small molecule cancer therapeutics.

### BACKGROUND OF THE INVENTION

Cell cycle regulation plays a critical role in neoplastic disease, as well as disease caused by non-cancerous, pathologically proliferating cells. In recent years, there have been major developments in the understanding of the cell cycle. Normal cell proliferation is tightly regulated by the activation and deactivation of a series of proteins that constitute the cell cycle machinery. The expression and activity of components of the cell cycle can be altered during the development of a variety of human diseases such as cancer, cardiovascular disease, psoriasis, where aberrant proliferation contributes to the pathology of the illness. There are genetic screens to isolate critical components for cell cycle regulation using different organisms such as yeast, worms, flies, etc., since cell cycle regulation is the most common machinery among all eukaryotic cells. However, there is a need to establish screening for understanding human diseases caused by disruption of cell cycle regulation. Identifying targets such as proteins and their ligands is important for developing therapeutic regents to treat cancer and other proliferative diseases.

### BRIEF SUMMARY OF THE INVENTION

The present invention therefore provides peptides, peptide 35/88, peptide 38, peptide 40, and peptide 41, that block the cell cycle of A549 cells. Binding partners of these peptides have been identified using binding affinity assay with biotinylated forms of the peptide and cellular extracts. Binding partners are identified using, e.g., MALDI-TOF.

Peptide 35 (also called peptide 88) has the amino acid sequence RLRRICSGILLIRRILGIFV, optionally with a vector-derived sequence RPVR or RPVRP at the C-terminus. Peptide 38 has the amino acid sequence TSGLLKLVQAKRKCCIS. Peptide 40 has the amino acid sequence RWDPTRLLRFRFLRMLVRRS, optionally with a vector-derived sequence RPVR or RPVRP at the C-terminus. Peptide 41 has the amino acid sequence GRGCIFRWRRGLRGMMRLFK. The peptides optionally have lysine residues fused to the N-terminus (e.g., 7 lysine residues).

The nucleotide sequences encoding the peptides are

Peptide 35/88:
CGGCTCCGGAGAATATGTAGCGGCATTCTGCTCATCCGTAGGATATTGGG
CATTTTCGTTAGGCCCGTGAGGCCCTAA
Peptide 38:
ACTAGTGGGTTGCTGAAGCTGGTGCAGGCTAAGCGTAAGTGTTGTATTAG
TTA
Peptide 40:
CGTTGGGATCCGACGCGATTGCTGCGATTTCGGTTCCTCCGGATGCTAGT
GAGGCGGAGTAGGCCCGTGAGGCCCTAA
Peptide 41:
GGAAGGGGATGTATCTTTCGATGGAGGAGAGGCCTGCGGGGAATGATGAG
ACTATTTAAGTAG

Binding partners of these peptides, or complexes of the binding partners, or the binding peptides themselves are targets for cancer drug development, e.g., for small molecule cancer therapeutics. These therapeutics can be used to treat diseases such as Hodgkin's lymphoma, gastric carcinoma, B-cell lymphoma, pancreatic cancer, breast cancer, lung cancer, prostate cancer, colorectal cancer, leukemia and lymphomas, and taxol-sensitive cancer.

In one embodiment, the interacting partners of each individual polypeptide, or a complex containing two or more binding partners, are used as drug targets for cancer therapeutics, e.g., tubulin, restin, actin, importin beta-1, annexin II, calpactin, and a complex of tubulin and any of the aforementioned polypeptides.

In one embodiment, peptide 41 or homologs or derivatives thereof is used as an anti-metastatic agent for treating cells surviving adriamycin chemotherapy or other chemotherapies.

In another embodiment, the peptides or their derivatives, or nucleic acids encoding the peptides or their derivatives, or the binding partners or nucleic acids encoding the binding partners can be used as cancer therapeutics.

In one embodiment, the invention provides a peptide comprising at least 14 amino acids, comprising at least 5 arginine residues, and having a motif X(2-3 aa)X′(2-3 aa) X″(1 aa)X′″, wherein X, X′, X″, and X′″ are individually large hydrophobic amino acid selected from the group consisting of L, I, F, M, Y, W, and wherein at least one is L or I.

### BRIEF DESCRIPTION OF THE DRAWINGS

FIG. 1 shows an SDS gel of the affinity extract for peptide 40 and its inactive mutant. This peptide blocks the cell cycle of A549 cells. Biotinylated peptide 40 is fused to the C-terminus of GFP with a linker EEAAKA (biotin-GMDELYK-EEAAKA-RWDPTRLLRFRFLRMLVRRSrpvr). Also tested is inactive a biotinylated alanine mutant biotin-GMDELYK-EEAAKA-RWDPTRALRARFARALVRRSrpvr. The difference bands were identified by MALDI-time of flight mass spectrometry. This peptide affinity extracts beta tubulin, importin beta 1 and 3 (and all or part of the nuclear pore complex), elongation factor tu, an ATP/ADP carrier protein, and a zinc finger protein.

FIG. 2 shows an SDS gel of the affinity extract for peptide 41 and its inactive mutant. This peptide blocks the cell cycle of A549 cells. Peptide 41 is fused to the C-terminus of GFP with a linker EEAAKA (GMDELYK-EEAAKA-GRGCIFRWRRGLRGMMRLFK). The difference bands were identified by MALDI-time of flight mass spectrometry. This peptide affinity extracts beta tubulin, portion beta subunits 1, 3, and 7, annexin II, calpactin/S-100, and chaperoninBip/GRP78.

FIG. 3 shows an SDS gel of the affinity extract for peptide 35 and its inactive mutant. This peptide blocks the cell cycle of A549 cells. The difference bands were identified by MALDI-time of flight mass spectrometry. This peptide affinity extracts beta-tubulin, actin, importin beta, and restin.

FIG. 4 shows the activity of Peptides 40, 41 and alanine mutants of Peptides 401 and 41 when incubated with A549 cells.

FIG. 5 shows nucleotide and amino acid sequences of #38, #40, #41, and #88. The amino acid sequences and the in-frame stop codon in each peptide are indicated as bold letters. The vector sequence is underlined.

FIG. 6 shows cell tracker assays for four anti-proliferative peptides, #38, #40, #41, and #88. Fluorescent intensity of GFP and Dil in A549.tTA cells expressing #38, #40, #41, #88, dsGFP or C-terminal peptide of the CDK inhibitor p21 was analyzed with MoFlo. Dil intensity of GFP positive population (solid line), GFP high population (dashed line), and GFP negative population (dotted line) is shown as histograms (middle, right). Gates for three populations are indicated in GFP histograms (left).

FIG. 7 shows cell cycle analysis for four anti-proliferative peptides, #38, #40, #41, and #88. Fluorescent intensity of GFP and Hoechst 33258 in AS49.tTA cells expressing #38, #40, #41, #88, or dsGFP was analyzed with MoFlo. Cell cycle profile of GFP low population (R1), GFP high population (R2), and GFP negative population (Rn) is shown as histograms. Gates for three populations are indicated in GFP histograms (left).

FIG. 8 shows variants of synthetic #40 and 41. The peptides with seven Lys and linker (GGEEAAKA) in N-terminus were synthesized (New England Peptide Inc, K7_{—}#40, K7_{—}#40M, K7_{—}#41, and K7_{—}#41M). Mutated residues were underlined.

FIG. 9 shows anti-proliferative effect of synthetic peptides. K7_{—}#40 K7_{—}#40M, K7_{—}#41, and K7_{—}#41M. A549 cells were incubated with the peptides at a final concentration ranging from 0.8 to 25 uM in triplicate for 24 hours. The number of nuclei and apoptotic nuclei in each well was counted using a Zeiss Axiovert microscope, UV filter set and Photometrics camera. The mean percentage of apoptotic nuclei in triplicate is shown. Closed square; K7_{—}#40, closed diamond; K7_{—}#40M, closed circle; K7_{—}#41, closed triangle; K7_{—}#41M.

FIG. 10 shows the effect of lys7-peptides on A549 lung carcinoma cells. Lys7-peptide 40, 41, or inactive tetra-alanine mutants of each were added to the culture medium of the cells at increasing doses, and different assays run. Effect of added peptides on the number of cell nuclei observed, for a fixed number of cells, 48 hours after exposure to the peptide. A Cellomics high content screening system was used to observe cells at high resolution. Filled circles represent the active peptide 41 sequence; filled squares represent the active peptide 40 sequence; open circles represent inactive mutant peptide 41; open squares represent inactive mutant peptide 40; open blue circles represent untreated cells. Values were averages of triplicate measurements+one standard deviation. Peptides 40 and 41 caused a dose-dependent decrease in the number of observed cell nuclei to ca. 10% of original values.

FIG. 11 shows the effect of peptides on apoptosis of A549 cells after a 48 hour exposure to varying doses of peptides. Symbols are as in B above. Peptides 40 and 41 both cause a dose-dependent increase in apoptosis not seen with their corresponding mutant peptides.

FIG. 12 shows the effect of lys₇-peptide 41 on the cell cycle of A549 cells after 48 hours exposure. Top: active peptide 41, bottom: mutant peptide 41. Cells are in G1 phase (diamonds), S phase (squares), G2 phase (triangles) or M phase (circles). The active peptide causes an increase in cells in M phase; cells in G2 or S phase are depleted. The cells seen here have not undergone apoptosis.

FIG. 13 shows the effect of lys7-peptide 40 on the cell cycle of A549 cells after 48 hours exposure. Top: active peptide 40, bottom: mutant peptide 40. Symbols are as in C above. The active peptide causes a large increase in cells in M phase; while all other phases are depleted.

FIG. 14 shows biotin-peptide affinity extracts examined by 1D SDS-PAGE and Western blotting. Lysates of ca. 10⁸ A549 cells, treated with a protease inhibitor cocktail, were affinity extracted with biotin-GMDELYKEEAAKA-peptide 40, 41 and 35 in separate experiments. Peptides with the four leucine-rich motif residues mutated to alanine, which were inactive, were used as controls. Bands present at higher levels in the active peptide affinity extract compared to the mutant peptide extract are labeled. Each peptide extracted 5-6 noticeable bands, some of which were common to more than one peptide extract. The largest band, 4, was identified as beta-tubulin by MALDI-TOF mass spectrometry, band 2 was identified as an importin beta subunit.

FIG. 15 shows western blots of proteins present in A549 cell affinity extracts of peptides 35, 40 and 41. Adjacent lanes consist of biotinylated peptides and biotinylated inactive mutant peptides. Lanes 1 and 8: cell lysate and positive control proteins when available; lanes 3, 5 and 7: peptides 35, 40 and 41; lanes 2, 4 and 6: inactive mutant peptides 35, 40 and 41. Western blot confirming some proteins found by mass spectrometry. Elongation factor tu was confirmed in extracts of peptide 41 and perhaps peptide 40; calpactin and PCNA are present in all three peptide extracts. Annexin II, HSP70, and the chaperonin BiP are differentially present in the peptide 41 extract but not the mutant controls. HSP 70 is present in all other peptide extracts and controls.

FIG. 16 shows a western blot of proteins associated with the nuclear pore complex or nucleocytoplasmic transport. Importin beta (containing 2-3 bands) was differentially present in all three peptide extracts, as was nucleoporin p62, importin alpha, ran, and exportin 1. RCC1 was present in the peptide 35 extract but missing from the controls.

FIG. 17 shows LC/MS/MS analysis of trypsin-digested entire affinity extracts overall identified the most interacting proteins, and the most unique proteins. Western blotting, and MALDI-TOF mass spectrometry of in gel digests of silver stained difference gel bands, also identified a unique proteins.

FIG. 18 shows that the three nuclear excluded peptides extracted 5 main sets of interacting proteins, indicated in the boxes above. These sets of proteins have linked functions; one common element is interactions with nucleocytoplasmic transport system proteins indicated in the gray box, suggesting that the primary effect of the peptides is to bind one or more of these elements.

### DETAILED DESCRIPTION OF THE INVENTION INTRODUCTION

The four peptides described herein, peptide 35, 38, 40, and 41, are functional when fused to GFP for blocking the cell cycle of A549 cells. The biotinylated forms of these peptides were used to identify and purify binding partners, using affinity extraction. Peptide 40 binds to beta tubulin, importin beta 1 and beta 3, elongation factor tu, and ATP/ADP carrier protein, and a zinc finger protein. The presence of a prominent tubulin band suggests that a complex involving tubulin is a binding partner for this peptide in blocking the cell cycle. This complex may include all or part of the nuclear pore complex, as importin beta 1 and 3 are also extracted (Cell. Strc. Func. 24:425 (1999)). Tubulin is a target of anti-cancer molecules such as taxol. Peptide 40 may also be used as a therapeutic or a lead compound for the development of a drug that binds to the same site of tubulin or a complex including tubulin.

Peptide 41 binds to beta tubulin, importin beta subunits 1, 2, and 7, annexin II, calpactin/S-100, and the chaperonin BiP/GRP78. Again, tubulin or a tubulin-bound complex may be binding partners of this peptide in blocking the cell cycle. Protein tyrosine kinase Annexin II is involved in mitogenic signal transduction, DNA synthesis (as part of the primer recognition complex) cell proliferation, adhesion, and membrane fusion (Mol. Cell. Biochem. 199:139-147 (1999); J. Cell. Sci. 101:35-41 (1992). Annexin II is over-expressed in gastric carcinoma, B lymphomas, and pancreatic cancers (Anticancer Res. 21:1339-1345 (2001); Biochim Biophys Acta 1313:295-301 (1996); Carcinogenesis 14:2575-2579 (1993)). Antisense oligos to annexin II reduce DNA synthesis and S to G2 cell cycle progression (J. Cell. Sci. 101:3541 (1992)). Downregulation of annexin II with an antisense molecule blocks cell division and proliferation in 293 cells (Mol. Cell. Biochein. 199:139-147 (1999)). Annexin II overexpression is also correlated with metastatic progression in cells surviving adriamycin chemotherapy (Clin. Exp. Metastasis 18:45-50 (2000)).

Peptide 35 binds to beta-tubulin, actin, importin beta, and restin. Restin is an intermediate filament-associated protein whose yeast homolog (bik1p) binds microtubules and TOR, the target of rapamycin. TOR regulates microtubule structure and function, possibly through the yeast homolog of restin, bik1p (Curr. Biol. 10:861-864 (2000)). Peptide 35 may affect microtubule structure and function. This peptide may trigger a cell cycle checkpoint brought on by microtubule abnormalities caused by the peptide. Restin is thought to act as a liker protein between microtubules and endosomes (Genomics 53:348-358 (1998)). Restin is highly expressed in Hodgkin's disease and anaplastic large-cell lymphomas and may be a contributing factor in the progression of Hodgkin's disease (Blood 80:2891-2896 (1991); EMBO J. 11:2103-2113 (1992)).

The binding partners identified herein therefore represent drug targets for compounds that suppress or activate cellular proliferation, or cause cell cycle arrest, or cause release from cell cycle arrest. Agents identified in these assays, including small organic molecules, peptides, cyclic peptides, nucleic acids, antibodies, antisense nucleic acids, and ribozymes, that modulate cell cycle regulation and cellular proliferation via modulation of one of the binding partners described herein, can be used to treat diseases related to cellular proliferation, such as cancer.

Such modulators are useful for treating cancers, such as Hodgkin's lymphoma, gastric carcinoma, B-cell lymphoma, pancreatic cancer, melanoma, breast, ovarian, lung, gastrointestinal and colon, prostate, and leukemia and lymphomas, e.g., multiple myeloma. In addition, such modulators are useful for treating noncancerous disease states caused by pathologically proliferating cells such as thyroid hyperplasia (Grave's disease), psoriasis, benign prostatic hypertrophy, neurofibromas, atherosclerosis, restenosis, and other vasoproliferative disease.

Definitions

By “disorder associated with cellular proliferation” or “disease associated with cellular proliferation” herein is meant a disease state which is marked by either an excess or a deficit of cellular proliferation. Such disorders associated with increased cellular proliferation include, but are not limited to, cancer and non-cancerous pathological proliferation.

The phrase “functional effects” in the context of assays for testing compounds that modulate activity of binding partner of peptide 40, 41, or 35 as disclosed herein, includes the determination of a parameter that is indirectly or directly under the influence of the binding partner, e.g., an indirect, phenotypic or chemical effect, such as the ability to increase or decrease cellular proliferation, apoptosis, cell cycle arrest; or e.g., a direct, physical effect such as ligand binding or inhibition of ligand binding. A functional effect therefore includes ligand binding activity, the ability of cells to proliferate, apoptosis, and enzyme activity. “Functional effects” include in vitro, in vivo, and ex vivo activities.

By “determining the functional effect” is meant assaying for a compound that increases or decreases a parameter that is indirectly or directly under the influence of a binding partner disclosed herein, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for the protein; measuring inducible markers or transcriptional activation of the protein; measuring binding activity or binding assays, e.g. binding to antibodies; measuring changes in ligand binding activity; measuring cellular proliferation; measuring apoptosis; measuring cell surface marker expression; measurement of changes in protein levels for binding partner-associated sequences; measurement of RNA stability, phosphorylation or dephosphorylation; identification of downstream or reporter gene expression (CAT, luciferase, β-gal, GFP and the like), e.g., via chemiluminescence, fluorescence, calorimetric reactions, antibody binding, and inducible markers.

“Inhibitors”, “activators”, and “modulators” refer to activating, inhibitory, or modulating molecules identified using in vitro and in vivo assays of a binding partner disclosed herein. Inhibitors are compounds that, e.g., bind to, partially or totally block activity, decrease, prevent, delay activation, inactivate, desensitize, or down regulate the activity or expression of a binding partner disclosed herein, e.g., antagonists. “Activators” are compounds that increase, open, activate, facilitate, enhance activation, sensitize, agonize, or up regulate expression or activity of a binding partner disclosed herein, e.g., agonists. Inhibitors, activators, or modulators also include genetically modified versions of a binding partner disclosed herein, e.g., versions with altered activity, as well as naturally occurring and synthetic ligands, antagonists, agonists, antibodies, peptides, cyclic peptides, nucleic acids, antisense molecules, ribozymes, small chemical molecules and the like. Such assays for inhibitors and activators include, e.g., expressing a binding partner disclosed herein in vitro, in cells, or cell membranes, applying putative modulator compounds, and then determining the functional effects on activity, as described above.

Samples or assays comprising a binding partner disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSLUM62 scoring matrix (see Henikoff & Henikoff, Proc. Natl. Acad. Sci. USA 89:10915 (1989)) alignments (13) of 50, expectation (E) of 10, M=5, N=−4, and a comparison of both strands.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (P), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)∝₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al, Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al, Immunology Today 4: 72 (1983); Cole et al, pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chaims of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al, Bio/Technology 10:779-783 (1992); Lonberg et al., Nature 368:856-859 (1994); Morrison, Nature 368:812-13 (1994); Fishwild et al, Nature Biotechnology 14:845-51 (1996); Neuberger, Nature Biotechnology 14:826 (1996); and Lonberg & Huszar, Intern. Rev. Immunzol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al, Biotechnology 10:779-783 (1992)). Antibodies can also be made bispecific, i.e., able to recognize two different antigens (see, e.g., WO 93/08829, Trauriecker et al, EMBO J. 10:3655-3659 (1991); and Suresh et al, Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a protein, polymorphic variants, alleles, orthologs, and conservatively modified variants, or splice variants, or portions thereof, can be selected to obtain only those polyclonal antibodies that are specifically immunoreactive a specific protein and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (Vols. 1-3, 1992); Lloyd, The Art, Science and Technology of Pharmaceutical Compounding (1999); and Pickar, Dosage Calculations (1999)).

Assays for Proteins that Modulation Cellular Proliferation

High throughput functional genomics assays can be used to identify modulators of cellular proliferation. Such assays can monitor changes in cell surface marker expression, proliferation and differentiation, and apoptosis, using either cell lines or primary cells. Typically, the cells are contacted with a cDNA or a random peptide library (encoded by nucleic acids). In one embodiment, the peptides are cyclic or circular. The cDNA library can comprise sense, antisense, full length, and truncated cDNAs. The peptide library is encoded by nucleic acids. The effect of the cDNA or peptide library on the phenotype of cellular proliferation is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the peptide or with the protein encoded by the cDNA can be isolated using a affinity extract system, yeast two-hybrid system, mammalian two hybrid system, or phage display screen, etc. Targets so identified can be further used as bait in these assays to identify additional members of the cellular proliferation pathway, which members are also targets for drug development (see, e.g., Fields et al., Nature 340:245 (1989); Vasavada et al., Proc. Nat'l Acad. Sci. USA 88:10686 (1991); Fearon et al., Proc. Nat'lAcad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Suitable cell lines include A549 cells. Cell surface markers can be assayed using fluorescently labeled antibodies and FACS. Cell proliferation can be measured using ³H-thymidine or dye inclusion. Apoptosis can be measured using dye inclusion, or by assaying for DNA laddering or increases in intracellular calcium. Growth factor production can be measured using an immunoassay such as ELISA.

cDNA libraries are made from any suitable source. Libraries encoding random peptides are made according to techniques well known to those of skill in the art (see, e.g., U.S. Pat. Nos. 6,153,380, 6,114,111, and 6,180,343). Any suitable vector can be used for the cDNA and peptide libraries, including, e.g., retroviral vectors.

Isolation of Nucleic Acids

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

Nucleic acids, polymorphic variants, orthologs, and alleles can be isolated using nucleic acid probes and oligonucleotides under stringent hybridization conditions, by screening libraries. Alternatively, expression libraries can be used to clone the desired protein, polymorphic variants, orthologs, and alleles by detecting expressed homologs immunologically with antisera or purified antibodies.

To make a cDNA library, one should choose a source that is rich in the desired RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al, supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating a nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences directly from mRNA, from cDNA, from genomic libraries or cDNA libraries. Degenerate oligonucleotides can be designed to amplify homologs. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of mRNA in physiological samples, for nucleic acid sequencing, or for other purposes. Genes amplified by the PCR reaction can be purified from agarose gels and cloned into an appropriate vector.

Gene expression can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids can be used with high density oligonucleotide array technology (e.g., GeneChip™) to identify the protein of interest, orthologs, alleles, conservatively modified variants, and polymorphic variants in this invention. In the case where the homologs being identified are linked to modulation of cellular proliferation, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

A cloned gene or cDNA is typically cloned into intermediate vectors before transformation into prokaryotic or eukaryotic cells for replication and/or expression. These intermediate vectors are typically prokaryote vectors, e.g., plasmids, or shuttle vectors.

Expression in Prokaryotes and Eukaryotes

To obtain high level expression of a cloned gene, one typically subclones the gene or cDNA into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing the desired protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one preferred embodiment, retroviral expression systems are used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., a Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

After the expression vector is introduced into the cells, the transfected cells are cultured under conditions favoring expression of the protein of choice, which is recovered from the culture using standard techniques identified below.

Purification of Polypeptides

Either naturally occurring or recombinant protein can be purified for use in functional assays. Naturally occurring protein can be purified, e.g., from human tissue. Recombinant protein can be purified from any suitable expression system.

The protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant protein is being purified. For example, proteins having established molecular adhesion properties can be reversible fused to the protein. With the appropriate ligand, protein can be selectively adsorbed to a purification column and then freed from the column in a relatively pure form. The fused protein is then removed by enzymatic activity. Finally, protein could be purified using immunoaffinity columns.

A. Purification of Protein from: Recombinant Bacteria

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, it is possible to purify protein from bacteria periplasm. After lysis of the bacteria, when the protein exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying Proteins

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatogaphy

The protein can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Assays for Modulators of Peptide Binding Partner Proteins

A. Assays

Modulation of a binding partner disclosed herein, and corresponding modulation of cellular, e.g., tumor cell, proliferation, can be assessed using a variety of in vitro and in vivo assays, including cell-based models. Such assays can be used to test for inhibitors and activators of a binding partner disclosed herein, and, consequently, inhibitors and activators of cellular proliferation. Such modulators are useful for treating disorders related to pathological cell proliferation. Modulators are tested using either recombinant or naturally occurring protein, preferably human protein.

Measurement of cellular proliferation modulation with a binding partner disclosed herein or a cell expressing such a protein, either recombinant or naturally occurring, can be performed using a variety of assays, in vitro, in vivo, and ex vivo, as described herein. A suitable physical, chemical or phenotypic change that affects activity, e.g., enzymatic activity, cell proliferation, or ligand binding can be used to assess the influence of a test compound on the polypeptide of this invention. When the functional effects are determined using intact cells or animals, one can also measure a variety of effects, such as, ligand binding, transcriptional changes to both known and uncharacterized genetic markers (e.g., northern blots), changes in cell metabolism, changes related to cellular proliferation, cell surface marker expression, DNA synthesis, marker and dye dilution assays (e.g., GFP and cell tracker assays), contact inhibition, tumor growth in nude mice, etc.

In Vitro Assays

Assays to identity compounds with modulating activity can be performed in vitro. Such assays can used full length protein or a variant thereof, or a fragment, such as an enzymatic domain. Purified recombinant or naturally occurring protein can be used in the in vitro methods of the invention. In addition to purified protein, the recombinant or naturally occurring protein can be part of a cellular lysate or a cell membrane. As described below, the binding assay can be either solid state or soluble. Preferably, the protein or membrane is bound to a solid support, either covalently or non-covalently. Often, the in vitro assays of the invention are ligand binding or ligand affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the protein or a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, antibodies, and ligand analogs. A wide variety of assays can be used to identify modulator binding, including labeled protein-protein binding assays, electrophoretic mobility shifts, immunoassays, enzymatic assays, and the like. In some cases, the binding of the candidate modulator is determined through the use of competitive binding assays, where interference with binding of a known ligand is measured in the presence of a potential modulator. Either the modulator or the known ligand is bound first, and then the competitor is added. After the protein is washed, interference with binding, either of the potential modulator or of the known ligand, is determined. Often, either the potential modulator or the known ligand is labeled.

Cell-Based In Vivo Assays

In another embodiment, protein is expressed in a cell, and functional, e.g., physical and chemical or phenotypic, changes are assayed to identify modulators of cellular proliferation, e.g., tumor cell proliferation. Cells expressing the proteins of interest can also be used in binding assays and enzymatic assays. Any suitable functional effect can be measured, as described herein. For example, cellular morphology (e.g., cell volume, nuclear volume, cell perimeter, and nuclear perimeter), ligand binding, kinase activity, apoptosis, cell surface marker expression, cellular proliferation, GFP positivity and dye dilution assays (e.g., cell tracker assays with dyes that bind to cell membranes), DNA synthesis assays (e.g., ³H-thymidine and fluorescent DNA-binding dyes such as BrdU or Hoescht dye with FACS analysis), are all suitable assays to identity potential modulators using a cell based system. Suitable cells for such cell based assays include both primary cancer or tumor cells and cell lines, as described herein, e.g., A549 (lung), MCF7 (breast, p53 wild-type), H1299 (lung, p53 null), Hela (cervical), PC3 (prostate, p53 mutant), MDA-MB-231 (breast, p53 wild-type). Cancer cell lines can be p53 mutant, p53 null, or express wild type p53. The protein can be naturally occurring or recombinant. Also, fragments or chimeric proteins can be used in cell based assays.

Cellular polypeptide levels can be determined by measuring the level of protein or mRNA. The level of protein is measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, protein expression can be measured using a reporter gene system. Such a system can be devised using a protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

Animal Models

Animal models of cellular proliferation also find use in screening for modulators of cellular proliferation. Similarly, transgenic animal technology including gene knockout technology, for example as a result of homologous recombination with an appropriate gene targeting vector, or gene overexpression, will result in the absence or increased expression of the protein. The same technology can also be applied to make knock-out cells. When desired, tissue-specific expression or knockout of the protein may be necessary. Transgenic animals generated by such methods find use as animal models of cellular proliferation and are additionally useful in screening for modulators of cellular proliferation.

Knock-out cells and transgenic mice can be made by insertion of a marker gene or other heterologous gene into an endogenous gene site in the mouse genome via homologous recombination. Such mice can also be made by substituting an endogenous gene with a mutated version of the gene, or by mutating an endogenous gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Exemplary Assays

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Soft agar growth or colony formation in suspension assays can be used to identify modulators. Typically, transformed host cells (e.g., cells that grow on soft agar) are used in this assay. For example, A549 cell lines can be used. Techniques for soft agar growth or colony formation in suspension assays are described in Freshney, Culture of Animal Cells a Manual of Basic Technique, 3^{rd} ed., Wiley-Liss, New York (1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with [³H]-thymidine at saturation density can be used to measure density limitation of growth. See Freshney (1994), supra. The transformed cells, when contacted with cellular proliferation modulators, regenerate a normal phenotype and become contact inhibited and would grow to a lower density.

Contact inhibition and density limitation of growth assays can be used to identify modulators which are capable of inhibiting abnormal proliferation and transformation in host cells. Typically, transformed host cells (e.g., cells that are not contact inhibited) are used in this assay. For example, A549 cell lines can be used. In this assay, labeling index with [³H]-thymidine at saturation density is a preferred method of measuring density limitation of growth. Transformed host cells are contacted with a potential modulator and are grown for 24 hours at saturation density in non-limiting medium conditions. The percentage of cells labeling with [³H]-thymidine is determined autoradiographically. See, Freshney (1994), supra. The host cells contacted with a modulator would give arise to a lower labeling index compared to control (e.g., transformed host cells transfected with a vector lacking an insert).

Growth Factor or Serum Dependence

Growth factor or serum dependence can be used as an assay to identify modulators. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When transformed cells are contacted with a modulator, the cells would reacquire serum dependence and would release growth factors at a lower level.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, tumor angiogenesis factor (OAF) is released at a higher level in tumor cells than their normal counterparts. See, e.g., Folkman, Angiogenesis and cancer, Sem Cancer Biol. (1992)).

Tumor specific markers can be assayed to identify modulators which decrease the level of release of these markers from host cells. Typically, transformed or tumorigenic host cells are used. Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

Invasiveness into Matrigel

The degree of invasiveness into Matrigel or some other extracellular matrix constituent can be used as an assay to identify modulators which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used as host cells. Therefore, modulators can be identified by measuring changes in the level of invasiveness between the host cells before and after the introduction of potential modulators. If a compound modulates a binding partner disclosed herein, its expression in tumorigenic host cells would affect invasiveness.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

Apoptosis Analysis

Apoptosis analysis can be used as an assay to identify modulators. In this assay, cell lines, such asA549, can be used to screen modulators. Cells are contacted with a putative modulator. The cells can be co-transfected with a construct comprising a marker gene, such as a gene that encodes green fluorescent protein, or a cell tracker dye. The apoptotic change can be determined using methods known in the art, such as DAPI staining and TUNEL assay using fluorescent microscope. For TUNEL assay, commercially available kit can be used (e.g., Fluorescein FragEL DNA Fragmentation Detection Kit (Oncogene Research Products, Cat.# QIA39)+Tetramethyl-rhodamine-5-dUTP (Roche, Cat. # 1534 378)). Cells contacted with modulators would exhibit, e.g., an increased apoptosis compared to control.

G₀/G₁ Cell Cycle Arrest Analysis

G₀/G₁ cell cycle arrest can be used as an assay to identify modulators. In this assay, cell lines, such as RKO or HCT116, can be used to screen modulators. The cells can be co-transfected with a construct comprising a marker gene, such as a gene that encodes green fluorescent protein, or a cell tracker dye. Methods known in the art can be used to measure the degree of G₁ cell cycle arrest. For example, a propidium iodide signal can be used as a measure for DNA content to determine cell cycle profiles on a flow cytometer. The percent of the cells in each cell cycle can be calculated. Cells contacted with a modulator would exhibit, e.g., a higher number of cells that are arrested in G₀/G₁ phase compared to control.

Tumor Growth In Vivo

Effects of modulators on cell growth can be tested in transgenic or immune-suppressed mice. Knock-out transgenic mice can be made, in which the endogenous gene is disrupted. Such knock-out mice can be used to study effects of a binding partner disclosed herein, e.g., as a cancer model, as a means of assaying in vivo for compounds that modulate a binding partner disclosed herein, and to test the effects of restoring a wild-type or mutant binding partner to a knock-out mice.

Knock-out cells and transgenic mice can be made by insertion of a marker gene or other heterologous gene into the endogenous gene site in the mouse genome via homologous recombination. Such mice can also be made by substituting the endogenous gene with a mutated version of the gene, or by mutating the endogenous gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987). These knock-out mice can be used as hosts to test the effects of various modulators on cell growth.

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportions of cases, while normal cells of similar origin will not. Hosts are treated with modulators, e.g., by injection. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, modulators which are capable, e.g., of inhibiting abnormal cell proliferation can be identified.

B. Modulators

The compounds tested as modulators of protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of the protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microciter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

C. Solid State and Soluble High Throughput Assays

In one embodiment the invention provides soluble assays using a binding partner disclosed herein, or a cell or tissue expressing such a protein, either naturally occurring or recombinant. In another embodiment, the invention provides solid phase based in vitro assays in a high throughput format, where the protein is attached to a solid phase substrate. Any one of the assays described herein can be adapted for high throughput screening.

In the high throughput assays of the invention, either soluble or solid state, it is possible to screen up to several thousand different modulators or ligands in a single day. This methodology can be used for proteins in vitro, or for cell-based or membrane-based assays comprising a protein of interest. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chent. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Immunological Detection of Polypeptides

In addition to the detection of gene and gene expression using nucleic acid hybridization technology, one can also use immunoassays to detect proteins of the invention. Such assays are useful for screening for modulators of cellular proliferation, as well as for therapeutic and diagnostic applications. Immunoassays can be used to qualitatively or quantitatively analyze protein. A general overview of the applicable technology can be found in Harlow & Lane, Antibodies: A Laboratory Manual (1988).

A. Production of Antibodies

Methods of producing polyclonal and monoclonal antibodies that react specifically with the desired proteins are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of a specific protein may be used to produce antibodies specifically reactive with protein. For example, recombinant protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an, immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against other proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular ortholog, such as a human ortholog, can also be made, by subtracting out other cross-reacting orthologs from a species such as a non-human mammal. In this manner, antibodies that bind only to the specific protein may be obtained.

Once the specific antibodies against the desired protein are available, the protein can be detected by a variety of immunoassay methods. In addition, the antibody can be used therapeutically as modulators. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

Protein can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the protein or antigenic subsequence thereof). The antibody may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled antigen or a labeled antibody. Alternatively, the labeling agent may be a third moiety, such a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting antigen in samples may be either competitive or noncompetitive. Noncompetitive immunoassays are assays in which the amount of antigen is directly measured. In one preferred “sandwich” assay, for example, the antibodies can be bound directly to a solid substrate on which they are immobilized. These immobilized antibodies then capture antigen present in the test sample. Proteins thus immobilized are then bound by a labeling agent, such as a second specific antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of protein present in the sample is measured indirectly by measuring the amount of a known, added (exogenous) protein displaced (competed away) from an antibody by the unknown protein present in a sample. In one competitive assay, a known amount of protein is added to a sample and the sample is then contacted with an antibody that specifically binds to the protein. The amount of exogenous protein bound to the antibody is inversely proportional to the concentration of specific protein present in the sample. In a particularly preferred embodiment, the antibody is immobilized on a solid substrate. The amount of specific protein bound to the antibody may be determined either by measuring the amount of specific protein present in protein/antibody complex, or alternatively by measuring the amount of remaining uncomplexed protein. The amount of specific protein may be detected by providing a labeled molecule.

A hapten inhibition assay is another preferred competitive assay. In this assay the known protein is immobilized on a solid substrate. A known amount of antibody is added to the sample, and the sample is then contacted with the immobilized antigen. The amount of antibody bound to the known immobilized antigen is inversely proportional to the amount of antigen protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, a protein can be immobilized to a solid support. Proteins are added to the assay that compete for binding of the antisera to the immobilized antigen. The ability of the added proteins to compete for binding of the antisera to the immobilized protein is compared to the ability of the protein to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a protein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein that is required to inhibit 50% of binding, then the second protein is said to specifically bind to the polyclonal antibodies generated to the immunogen.

Other Assay Formats

Western blot (immunoblot) analysis is used to detect and quantify the presence of a protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the protein. The antibodies specifically bind to the protein on the solid support. These antibodies may be directly labeled or alternatively may be subsequently detected using labeled antibodies (e.g., labeled sheep anti-mouse antibodies) that specifically bind to the antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:3441 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize the protein, or secondary antibodies that recognize the antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Pharmaceutical Compositions and Administration

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of the protein, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

### EXAMPLES

The following examples are offered to illustrate, but not to limit the claimed invention.

### Example 1

### Identification of Peptide Binding Partners

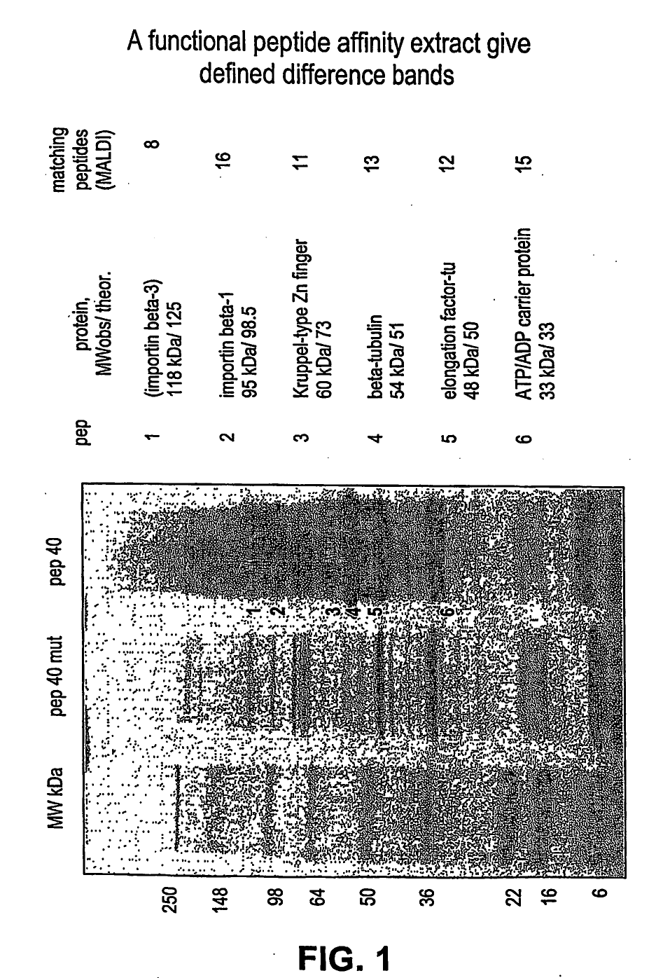

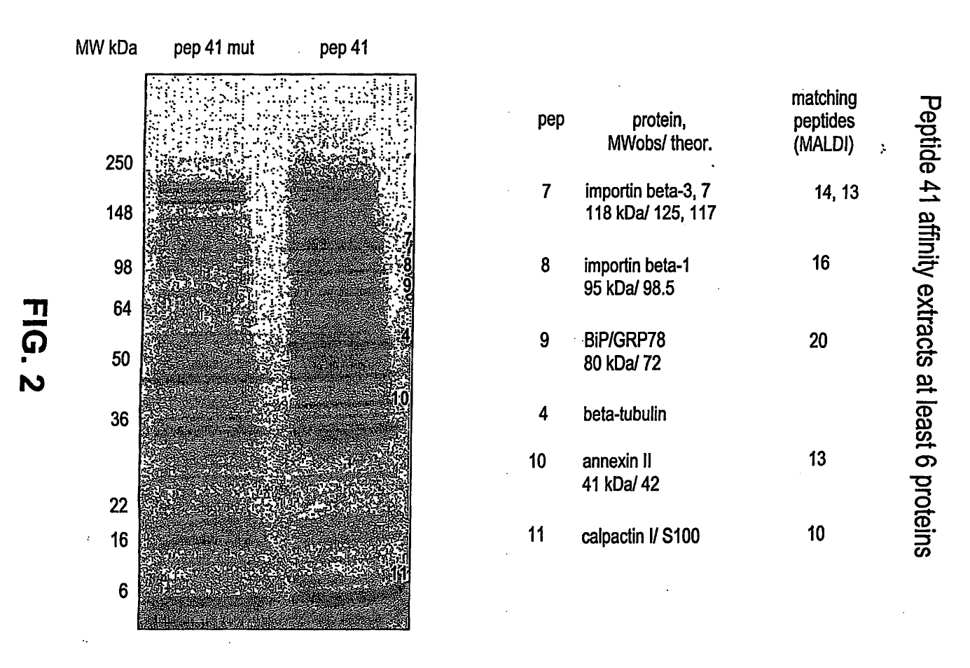

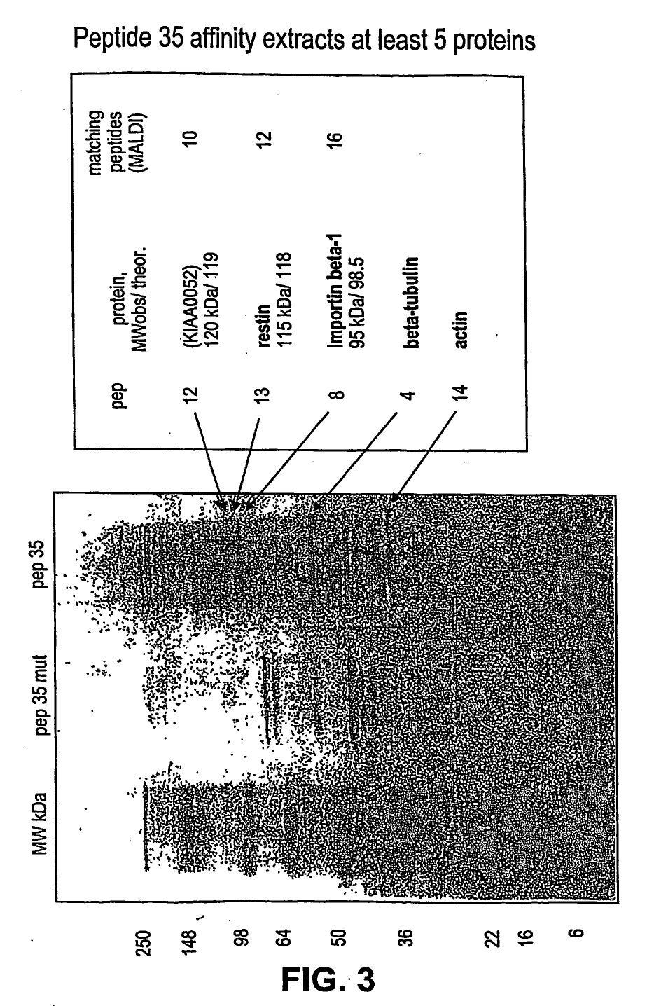

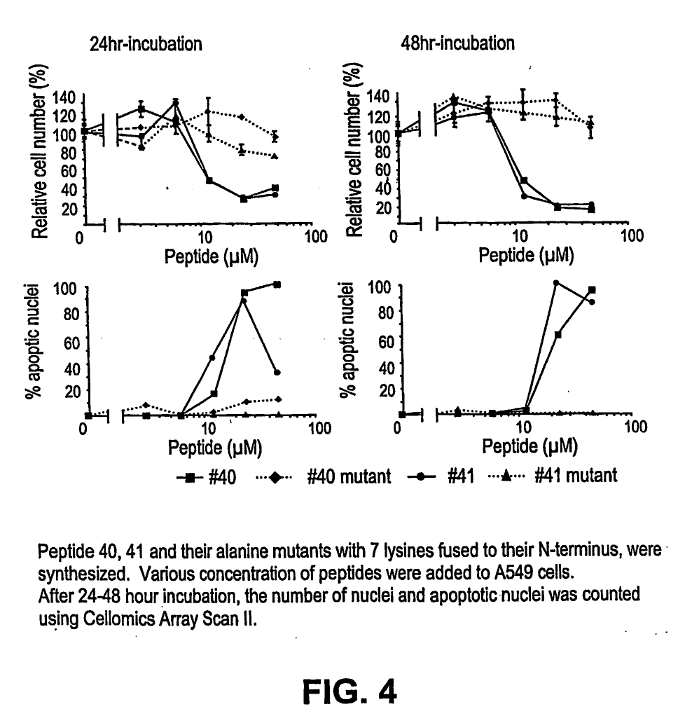

Peptides 35, 38, 40 and 41 were identified as peptides that had the ability to block the cell cycle of A549 cells. Binding partners of these peptides were identified using affinity extract assays. Biotinylated peptides were incubated with cellular extract, and the SDS gels were run to identify binding partners. Binding partners were further identified using MAILDI-TOF (see FIGS. 1-3). Mutant versions of the peptides were used as controls in the affinity assays.

### Example 2

### Cellular Localization and Anti-Proliferative Activity of Peptides

Introduction

Expression and activity of components of cell proliferation can be altered during development of a variety of human diseases such as cancer, cardiovascular disease, and psoriasis where aberrant proliferation contributes to pathology of the illness. Regulation of cell proliferation is highly conserved among all eukaryotic cells and tightly regulated by the activation and deactivation of series of proteins that constitute signaling pathways. These signaling pathways can be blocked by disruption of protein-protein interaction. Intracellular peptides can bind to specific surface of a protein and consequently block the interaction. Disrupting specific protein-protein interaction provides significant insights into cell biology and could lead to the development of novel therapeutics. Small peptides are a powerful tool for inhibiting intracellular signaling cascades as previously reported (Warbrick, E. et al., Curr Biol, 5:275-282 (1995); Picksley, S. M. et al., Oncogene, 9:2523-2529 (1994); Yuan, J. et al., Cancer Res, 62:4186-4190 (2002)). Treatment with peptides offers alternative approaches for therapy for several diseases including cancer (Yuan, J. et al., Cancer Res, 62:4186-4190 (2002)).

Cellular screens of random peptide libraries have been used to discover novel peptides with specific cellular phenotypic effects in yeast (Caponigro, G. et al., Proc Natl Acad Sci USA, 95:7508-7513 (1998); Geyer, C. R. et al., Proc Natl Acad Sci USA, 96:8567-8572 (1999); Norman, T. C. et al, Science, 285:591-595 (1999)) and in human cells following retroviral delivery of the library (Xu, X. et al, Nat Genet, 27:23-29 (2001); Peelle, B. et al., Chem Biol, 8:521-534 (2001); Kinsella, T. M. et al., J Biol Chem, 277:37512-37518 (2002)). There is a need to establish screens in human cells to discover novel elements of tumor cell growth in the context of their functional biology, which will increase understanding of the pathways, of the phenotypes being studied, and can allow discovery of novel drug targets. In addition, technologies for the rapid screening of peptides have also opened up many new avenues for the exploitation of peptide-based products in the field of drug discovery.

Protein scaffolds, including thioredoxin (Colas, P. et al., Nature, 380:548-550 (1996)), green fluorescent protein (GFP) (Peelle, B. et al., Chem Biol, 8:521-534 (2001); Abedi, M. R. et al, Nucleic Acids Res, 26:623-630 (1998)) and Staphylococcal nuclease (Norman, T. C. et al, Science, 285:591-595 (1999)) have been used for intracellular display in yeast, bacteria and mammalian cells. Peptide library members, presented in the context of a stable protein scaffold such as GFP, bind cellular macromolecules as part of their phenotypic effect. GFP in particular has a significant advantage because cellular localization and expression levels can be monitored with its fluorescence and GFP has low toxicity, high stability and no interaction by itself (Lorens, J. B. et al., Mol Ther, 1:438-447 (2000); Mattock, H. et al., Exp Cell Res, 265:234-241 (2001)).

A screen for growth inhibition using a GFP-fusion random peptide library is described below. The screen was based on detection of non-dividing cells with the cell tracker dye and reduction of cycling cells by infection of the retrovirus encoding diphtheria toxin alpha chain. Peptide identified from the screen showed unique cellar localization and significant anti-proliferative effect. The screen for growth inhibitors using a GFP fusion peptide library is a novel approach to identify and validate several functional peptide inhibitors, which may be not only a useful tool for understanding signaling pathways but also a therapeutic potential in the treatment of diseases including cancers.

Materials and Methods.

Screening of GFP Fusion C-Terminus 20mer Random Peptide Library

A retroviral peptide library consisting GFP fused at the C-terminus of a random 20mer peptide (GFP-C20) (1.1×109 independent inserts) was constructed as previously reported (Xu, X. et al, Nat Genet, 27:23-29 (2001); Peelle, B. et al., Chem Biol, 8:521-534 (2001)) in a tetracycline (tet) regulatable retroviral vector, TRA (Lorens, J. B. et al., Virology, 272:7-15 (2000)). The library was transfected into an amphotropic virus packaging cell line, ΦNX-Ampho, which produces infectious retrovirus up to 5×106 infectious units/ml, as described previously (Swift S, L. J. et al., Current Protocols in Immunology, 10.17.14-10.17.29 (1999)). 4×108 tetracycline regulatable transactivator (tTA) expressing lung tumor cells, A549.tTA cells (Lorens, J. B. et al., Virology, 272:7-15 (2000)) were infected with the supernatant from ΦNX-Ampho cells at 2500 r.p.m. for 1.5 hours at 32° C. Cells were placed for 12 hours at 32° C. Cells were then incubated at 37° C. for 24 hours to allow integration and expression of the retrovirus. We achieved 10% infection rate as determined by GFP fluorescence. Cells were then stained with a cell tracker dye, Dil (D-282, Molecular Probes, Inc., Eugene, Oreg. 97402, U.S.A.) for monitoring proliferation as per manufacturer's recommendation. Non-dividing cells remained Dil bright due to a decreased rate of cell division allowing fluorescence-activated cell sorting (FACS). Cells were infected with the retrovirus encoding diphtheria toxin alpha chain and sorted Dil bright and GFP positive cells with a fluorescence-activated cell sorter, MoFlo (Cytomation, Inc., Fort Collins, Colo. 80525, U.S.A.) in order to enrich non-dividing cells. Actively cycling cells were eliminated by retrovirally-delivered diphtheria toxin alpha chain from the population because they are susceptible to retroviral infection (Springett, G. M. et al., J Virol, 63:3865-3869 (1989); Batra, R. K. et al., Am J Respir Cell Mol Biol, 18:402-410 (1998)) while arrested cells remained intact. After another round of diphtheria toxin selection and cell sorting, 1.1×105 A549.tTA cells were plated into 384-well plates for single cell cloning in 30 μl media containing 10 μg/ml of a tetracycline analogue, doxycycline (Dox). More than 95% of tTA-mediated gene expression in A549.tTA cells was suppressed by 10 μg/ml Dox. 4128 colonies were picked and analyzed their growth in both Dox containing and Dox free media 95 clones were identified as clones that showed both reduction of cell growth in absence of Dox and normal cell growth in presence of Dox. Identified clones were expanded, total RNA was extracted, and cDNAs encoding peptide library elements were rescued using RT-PCR (AMV reverse transcriptase from Promega, Wis. 53711, U.S.A. and Vent DNA polymerase from New England Biolabs, Inc., MA 01915, U.S.A.). cDNAs were sequenced and re-ligated into the TRA vector. Naïve A549.tTA cells were infected with retrovirus encoding rescued peptides and confirmed the anti-proliferative effect of the peptides.

Cell Tracker Assay

A549.tTA cells were infected with tet-regulatable retroviruses expressing GFP fusion peptides, stained with the cell tracker dye, Dil, and incubated at 37° C. for five days. After the incubation, fluorescent intensity of GFP and Dil was analyzed with MoFlo. A growth ratio was obtained as the ratio of mean Dil fluorescent intensity for the GFP positive population relative to that of the GFP negative population.

Cell Cycle Analysis

A549.tTA cells expressing GFP fusion peptides were fixed with Cell Fixation/Permeabilization Kits (BD bioscience, Lexington, Ky., U.S.A.) as manufacturer's recommendation and stained with 5 mM Hoechst 33258 (Sigma, St. Louis, Mo., U.S.A.). GFP and Hoechst fluorescent intensity was analyzed with MoFlo.

Immunohistochemical Staining

A549.tTA cells expressing GFP fused peptides, #38 and #41, were plated on coverslips, washed with phosphate buffer saline, PBS, and fixed in 50% methanol and 50% acetone for 5 min. Then cells were washed twice with PBS, incubated with PBS containing 0.5% saponine and 0.5% BSA for 20 min. After washing cells with PBS twice, cells were incubated in PBS containing 0.5% saponine and 0.5% BSA at room temperature for 1 h with goat anti Clathrin heavy chain antibody (Sigma) or mouse anti-vinculin antibody (Transduction laboratories), washed intensively, and then incubated with the secondary Alexa Fluor R 546-labled donkey anti goat IgG antibody (Molecular Probes) or Alexa Fluor R 546-labled goat anti mouse IgG antibody (Molecular Probes), and Alexa Fluor R 488-labled rabbit anti GFP antibody (Molecular Probes). Both primary and secondary antibody solutions were precleared by centrifugation. After staining, the coverslips were mounted in Fluoromount-G (Fisher Scientific, Pittsburgh, Pa.). The samples were examined on a fluorescent microscope, Axiovert S100, (Carl Zeiss MicroImaging, Inc., Thornwood, N.Y., U.S.A.) equipped with a 63 Å˜1.4 numerical aperture oil immersion objective lens and the single fluorochrome filter sets for either Texas Red or fluorescein were used for visualization and recording of the images. The images were captured with a CCD camera, model C4742-95-12ER, (Hamamnatsu Photonics K.K., Hamamatsu, Japan).

High Content Imaging of Anti-Proliferative Peptides

A549 cells (American Type Culture Collection (ATCC), Manassas, Va., U.S.A.) were plated at 2000 cells/well in 96 well plates with F12K media supplemented with 10% fetal bovine serum and 1% penicillin/streptomycin and allowed to grow for 24 hours. Peptide dissolved in media was added to the cells to a final concentration ranging from 0.8 to 25 uM in triplicate. Following incubation with peptide for 24 hours, cells were fixed with 3.7% formaldehyde for 30 minutes, rinsed in PBS and stained with DAPI (Molecular Probes) to label nuclear DNA. Digital images were taken of the labeled nuclei in each well using a Zeiss Axiovert microscope, UV filter set and Photometrics camera. Images were analyzed using the Inage Pro software package to count the number of nuclei and measure the DNA intensity within each nucleus, and calculate the percentage of nuclei exhibiting the significant fragmentation characteristic of late apoptosis.

Results.

A Functional Screen for Antiproliferative Peptides.

Three techniques were incorporated and optimized to screen for peptides that inhibit tumor cell growth: 1) inducible expression system to isolate peptides that inhibit cell growth (tet-off inducible system), 2) assay for monitoring cells arrested or reduced their growth, 3) method to reduce cycling cells. Assay using cell tracker dye, Dil, was chosen for our screen because non-dividing cells or slow growing cells can be distinguished using a fluorescence-activated cell sorter without fixing cells. A retrovirus whose infection requires cell proliferation was used. The retrovirus encoding diphtheria toxin alpha chain (DT-alpha) was created for reduction of cycling cells and shown to efficiently eliminate infected A549 cells (data not shown). A549.tTA cells were infected with a tet-inducible (tet-off) retroviral GFP fusion random 20mer peptide library (GFP-C20) and selected according to fluorescent intensity of Dil, and resistance against infection of the retrovirus encoding DT-alpha (see Materials and Methods). After two rounds of DT-alpha selection and cell sorting, cells were plated for single cell cloning and peptide expression was suppressed with Dox. During single cell cloning, spontaneously arrested cell and cells arrested by the effect of retrovirus insertion can be eliminated. 95 clones were identified as clones that showed both reduction of cell growth in the absence of Dox (peptide expression on) and normal cell growth in presence of Dox (peptide expression off). The cDNA inserts encoding peptides were recloned and transduced into A549.tTA cells to verify their effect based on the fluorescent intensity of Dil. Four peptides, #38, #40, #41, and #88 were identified as significant inhibitors of cell proliferation of A549.tTA cells.

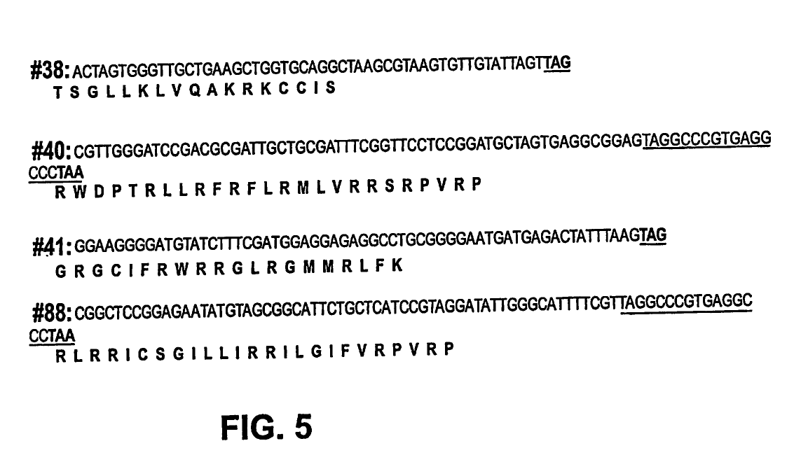

As shown in FIG. 5, the cDNA inserts of #38, #40, #41 and #88 encode 17mer, 24mer, 20mer, 24mer peptides, respectively. #40 and #88 have the additional amino-acid sequence (RPVRP) derived from the vector due to deletion of one nucleotide in the randomized region of the peptides.

BLAST search analysis revealed that the four peptides do not have more than 50% total identity to known sequences. #40, #41, and #88 are significantly rich in arginine and hydrophobic amino acids such as Leucine, Isoleucine, Methionine, and Phenylalanine (FIG. 5). Occurrence of Arginine, Leucine, Isoleucine, Methionine, and Phenylalanine in #40, #41, #88 is significantly higher than that in 41 peptides randomly picked from the library or theoretical occurrence of these amino acids (data not shown). Interestingly, the sequence of #40, #41, and #88 could be aligned with a leucine-rich motif found in HIV-1 Rev, HTLV-1 Rex, Mitogen-activated protein kinase kinase 1, MAPKK, Human homologue of mouse double minute 2, Hdm-2, or Protein kinase inhibitor, PKI, when other hydrophobic residues such as Isoleucine, Methionine, Phenylalanine and Tryptophan are allowed to replace some leucines (Table 1). The leucine-rich motifs are characterized by three leucines separated by 2-3 other residues, and a fourth leucine separated by a single residue, and are recognized by a complex including exportin-1 for transport out of the nucleus (Henderson, B. R. et al., Exp Cell Res, 256:213-224 (2000); Chook, Y. M. et al., Curr Opin Struct Biol, 11:703-715 (2001)). #38 contains a CAAX box motif associated with its membrane localization (Schaber, M. D. et al., J Biol Chem, 265:14701-14704 (1990)). CAAX box containing proteins need to be farnesylated or geranylgeranylated at their C-terminal end at their CAAX box after their synthesis (Fu, H. W. et al., Recent Prog Horm Res, 54:315-342; discussion 342-313 (1999)). The C-terminal half of #38 are nearly identical to the C-terminal 17 residues of cell division cycle 42 isoform 2, (CDC42C) (gi|6357472|ref|NP_{—}426359.1|) indicated in Table 2.

TABLE 1

Effect of GFP fusion peptides and their mutants

| | | Growth Ratio |
|---|---|---|
| Peptide | Sequence | (mean ± SE) |
| GFP-random 20 | | 1.1 ± 0.1 |
| mer fusion | | |
| dsGFP | 1.1 ± 0.1 | |
| p21C | KRRQTSMTDFYHSKRRLIFSKRKP | 4.5 ± 0.8 |
| #40 | RWDPTRLLRFRFLRMLVRRSRPVRP | 2.4 ± 0.3 |
| #40(M15A) | RWDPTRLLRFRFLRALVRRSRPVRP | 2.0 |
| #40(L13A/M15A) | RWDPTRLLRFRFARALVRRSRPVRP | 1.6 ± 0.2 |
| #40(F10A/L13A/M15A) | RWDPTRLLRARFARALVRRSRPVRP | 1.3 ± 0.1 |
| #41 | GRGCTFRWRRGLRGMMRAFK | 2.7 ± 0.2 |
| #41(L18A) | GRGCIFRWRRGLRGMMRAFK | 1.7 |
| #41(M16A/L18A) | GRGCIFRWRRGLRGMARAFK | 1.5 |
| #41(L12A/M16A/L18A) | GRGCIFRWRRGARGMARAFK | 1.1 ± 0.1 |
| #88 | RLRRICSGILLIRRILGIFVRPVRP | 2.7 ± 0.4 |
| #88(118A) | RLRRICSGILLIRRILGAFVRPVRP | 1.5 |
| #88(L16A/L18A) | RLRRICSGILLIRRIAGAFVRPVRP | 1.1 |
| #88(I12AILI6A/118A) | RLRRICSGILLARRIAGAFVRPVRP | 1.1 ± 0.1 |
| HIV1 REV | LPP-L-ERLTLD | |
| MAPKK | LQKKL-EELELD | |
| HTLV1 Rex | LSAQLYSSLSLD | |
| Hdm-2 | ISLSFDESLALC | |
| PKI | LALKL-AGLDIN | |
| #40 | RWDPTRLLR-F-RFLRMLVRRSRPVRP | |
| #41 | GRGCIFRWRRGLRGMMRLFK | |
| #88 | RLRRICSGILL-IRRILGIFVRPVRP | |

Effect of GFP fused peptides and their mutants. GFP fused peptides (#40, #41, #88 and their mutants) expressing A549.tTA cells were stained with Dil and fluorescent intensity of GFP and Dil was examined with MoFlo. A growth ratio was obtained as described above. Mutated residues were underlined. Amino acid sequences of #40, #41, and #88 were aligned with leucine-rich motifs of HIV-1 Rev (amino acid position 75 to 84, gi|16118296|gb|AAL12672.1|), HTLV-1 Rex (amino acid position 82 to 93, gi|7963889|gb|AAF71372.1), Mitogen-activated protein kinase kinase 1, MAPKK, (amino acid position 33 to 43, gi|5579478|ref|NP_{—}002746.1|), Human homologue of mouse double minute 2, Hdm-2, (amino acid position 190 to 200, gi|4505137|ref|NP_{—}002383.1|), and Protein kinase inhibitor, PKI (amino acid position 38 to 48, gi|203271|gb|AAA40867.1|).

TABLE 2

Effect of GFP fusion peptides and their mutants

| | | Growth Ratio |
|---|---|---|
| Peptide | Sequence | (mean ± SE) |
| GFP-random | | 1.1 ± 0.1 |
| 20mer fusion | | |
| dsGFP | | 1.1 ± 0.1 |
| p21C | KRRQTSMTDFYHSKRRLIFSKRKP | 4.5 ± 0.8 |
| #38 | TSGLLKLVQAKRKCCIS | 2.5 ± 0.2 |
| CDC42C | AALEPPETQPKRKCCIF | 1.3 ± 0.1 |
| #38NΔ(1-8) | QAKRKCCIS | 1.5 ± 0.1 |
| #38NΔ(1-13) | CCIS | 1.2 ± 0.1 |
| #38(T1A) | ASGLLKLVQAKRKCCIS | 3.5 ± 0.2 |
| #38(S2A) | TAGLLKLVQAKRKCCIS | 5.5 ± 0.8 |
| #38(G3A) | TSALLKLVQAKRKCCIS | 2.7 ± 0.2 |
| #38(L4A) | TSGALKLVQAKRKCCIS | 2.2 ± 0.1 |
| #38(L5A) | TSGLAKLVQAKRKCCIS | 1.9 ± 0.2 |
| #38(K6A) | TSGLLALVQAKRKCCIS | 1.4 ± 0.1 |
| #38(L7A) | TSGLLKAVQAKRKCCIS | 2.0 ± 0.2 |
| #38(V8A) | TSGLLKLAQAKRKCCIS | 2.7 ± 0.2 |
| #38(C14A) | TSGLLKLVQAKRKACIS | 1.1 ± 0.1 |

Effect of GFP fused peptides and their mutants. A549.tTA cells were infected with retroviruses encoding GFP fusion peptides (#38 and its mutants) and stained with the cell tracker dye, Dil. After the infection, fluorescent intensity of GFP and Dil was examined with MoFlo. A growth ratio was obtained as the ratio of mean Dil fluorescent intensity for the GFP positive population relative to that of the GFP negative population and data are shown as mean±SE in duplicate. Mutated residues or non-identical residues of the C-terminus of cell division cycle 42 isoform 2, CDC42C, (amino acid position 183 to 191, gi|16357472|ref|NP_{—}426359.1|) to #38 were underlined. As controls, entire GFP-C20 library, dsGFP and GFP fused p21C expressing A549.tTA cells were used.

Four Peptides Block Cell Growth and can Cause Cell Cycle Arrest.

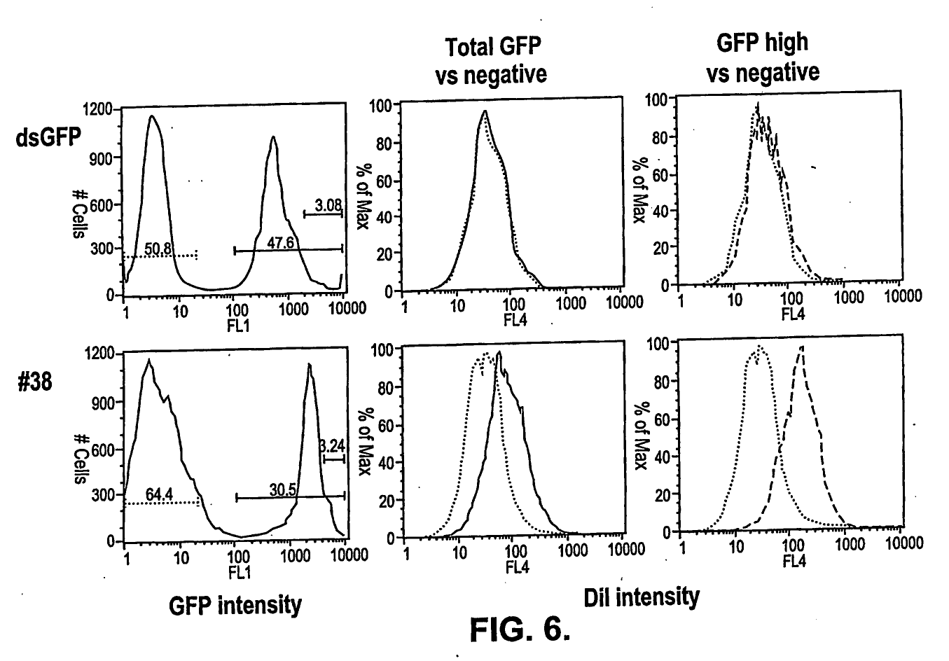

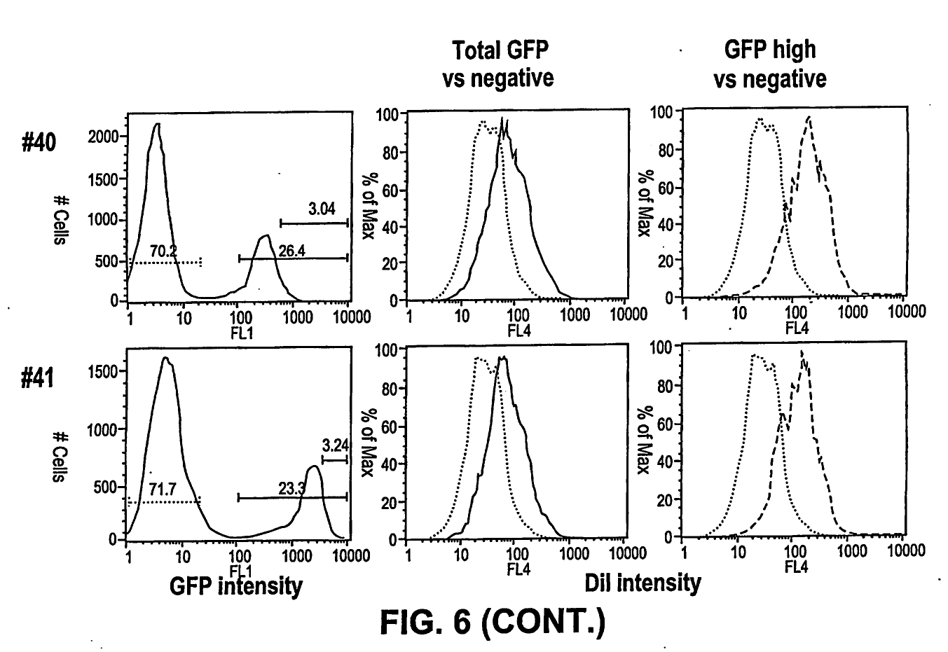

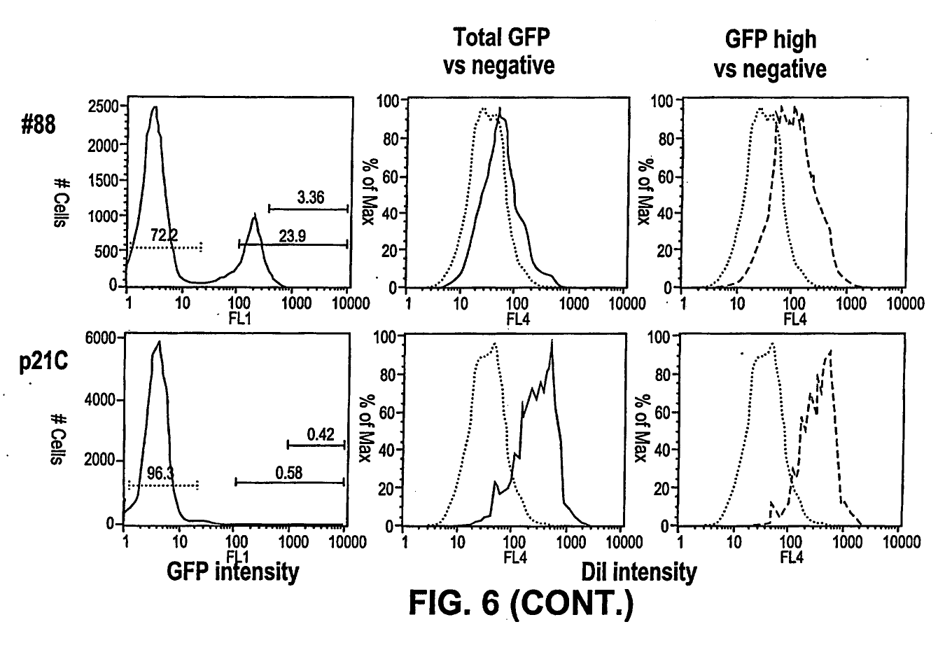

Since four peptides were isolated from the screen that is dependent upon fluorescent intensity of cell tracker dye, we performed cell tracker assay using Dil for their effect on cell proliferation (FIG. 6). Dil is a stable lipophilic dye that stains the cell membrane. Non-dividing cells or slow growing cells remain Dil bright due to the decreased rate of cell division. Fluorescent intensity of DiR in GFP positive population or GFP high population was compared with that in GFP negative population. There was no significant difference in Dil fluorescent intensity between the de-stabilized GFP (dsGFP) expressing population and the GFP negative population. The GFP highly positive population of the dsGFP expressing cells might have slight difference in Dil intensity compared with the GFP negative population because of its own toxicity to cells. Cells expressing proteins such as GFP-fused inactive peptide, #10, which is isolated from the screen as a negative control, or the entire GFP-C20 library, show an overlap in Dil fluorescence for the GFP positive and negative populations (data not shown). Since cells infected with the GFP-fused proliferating cell nuclear antigen (CNA)-binding C-terminal peptide of the tumor suppressor p21 (p21C) showed significant antiproliferative effect, a GFP-fused p21C was used as a positive control for cell tracker assay. GFP-fused p21C infected cells show higher Dil fluorescence in the GFP positive as compared to the negative population. Cells infected with retrovirus containing each of the four peptides showed higher Dil fluorescence for the GFP positive population, consistent with inhibition of cell proliferation. We did not observe massive cell death after infection of retrovirus encoding GFP fused peptides, #38, #40, #41, and #88 (data not shown). The anti-proliferative effect of the four peptides was more potent in the GFP high population. Based on GFP FACS analysis, two of the peptides, #40 and #88, were expressed at 10-fold lower levels than the other two peptides. All four peptides are similarly anti-proliferative in Hela cells (American Type Culture Collection (ATCC), Manassas, Va., U.S.A.), Colo205 cells (ATCC), H1299 (ATCC), MCF7(ATCC), and normal human mammary grand epithelial cells (HMEC) (CAMBREX, Baltimore, Md., U.S.A.) (data not shown).

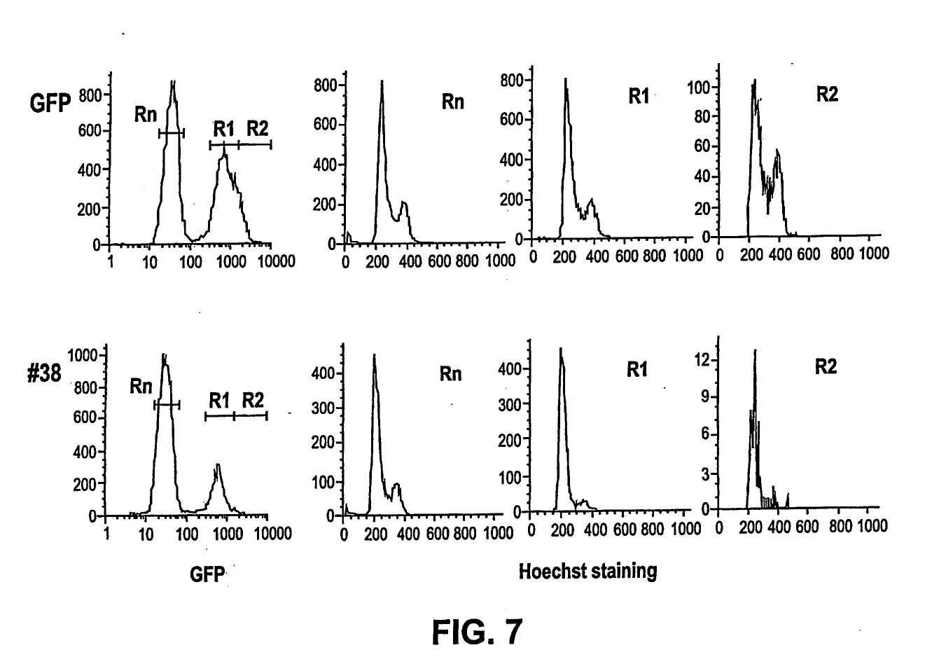

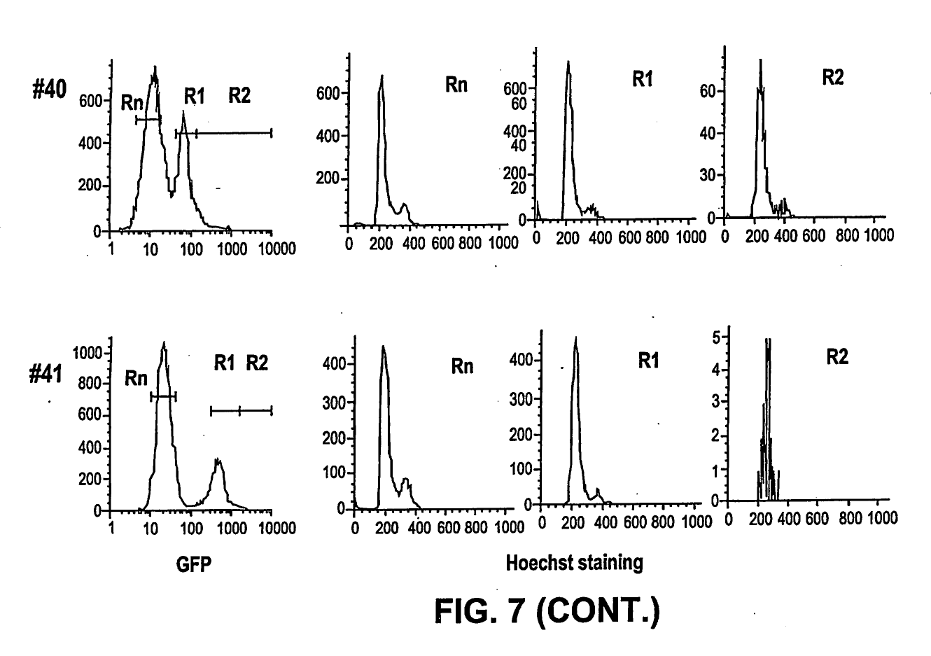

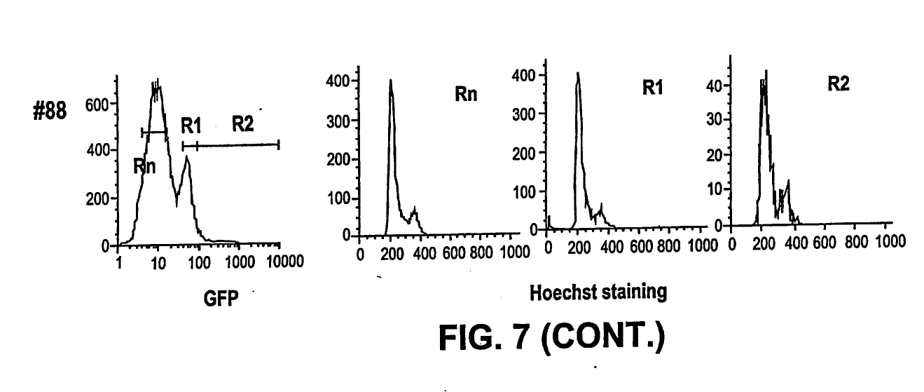

The effects of the four peptides on the cell cycle of A549 cells were analyzed. Cells expressing each peptide or dsGFP were stained with Hoechst 33258 to allow estimation of DNA content. The cell cycle profiles of GFP low population (R1), GFP high population (R2) and GFP negative population (Rn) are shown in FIG. 7. Cells expressing low level of dsGFP(R1) had a Hoechst profile that were similar to that of GFP negative cells, while the GFP high positive cells showed slight accumulation of cells in G2/M. Cells infected with either #38 or #41 appeared to be arrested in G0/G1 since cells in the S and G2/M phases were significantly reduced in both R1 and R2. Cells infected with either #40 or #88 showed no significant change in the DNA content profile in R1 nor R2 population.

Cellular Localization of Individual Peptides.

Since the peptides active in the functional screen were fused to GFP, their localization in A549.tTA cells was examined using a con-focal microscope. The four anti-proliferative peptides were predominantly localized to the cytoplasm in contrast with GFP localization of inactive peptide, #10 or dsGFP. #38. #40 and #41 were concentrated at the plasma membrane and granular cytoplasmic structures. The majority of #88 was localized to cytoplasmic granules but not the plasma membrane. #38 and #88 were clearly nuclear excluded. #40 and #41 were similarly nuclear excluded under a fluorescent microscope, however confocal images indicated that nuclear exclusion was not significant. There was GFP fluorescence from the nucleus in #40 and #41 expressing cells.

A co-localization study was performed with GFP fused peptides #38 and #41. GFP fusion #38 and #41 have similar localization to the plasma membrane, cytoplasmic granular structures, and the cell-cell contact area. Several membrane-localized proteins were examined. Analysis revealed that clathrin is localized to the plasma membrane and cytoplasmic granular structures and colocalizes to #38 and #41. We did not see significant co-localization of #38 and #41 with mitochondrial markers, golgi markers, actin, tublin, annexin II, vinculin, plakoglobin, desmoplakin nor Ras (data not shown except for vinculin). #41 contained a leucine-rich motif similar to a leucine-rich nuclear exclusion motif, which is recognized by exportin 1. However we did not observe colocalization of #38 and #41 with Exportin-1 and localization of both peptides was resistant to Leptomycin B, which inhibits Exportin-1's function (data not shown). Labeling with the #38 and #41 expressing cells with transferrin gave a different pattern than cellular localization of GFP fused #38 and #41 (data not shown).

#38 has a CAAX box motif in the C-terminus. A 15-carbon farnesyl or 20-carbon geranylgeranyl isoprenoid is linked to the Cys in a CAAX box motif by Farnesyl transferase or Geranylgeranyl transferase, respectively (Fu, H. W. et al., Recent Prog Horm Res, 54:315-342; discussion 342-313 (1999)). The role of farnesylation in cellular localization of #38 was examined. A549.tTA cells expressing #38 and #41 were treated with a peptidomimetic farnesyltransferase specific inhibitor, L-744,832 for 48 hours. As shown FIG. 6, cellular localization of #38, but not #41, was completely abolished in the presence of 2 μM L-744,832, indicating that localization of #38 is farnesyltransferase dependent. Interestingly localization of the C-terminal 17 residues of the CAAX box peptide from CDC42 (CDC42C) was also resistant to L-744,832 (data not shown).

Mutational Analysis of Four Peptides.

In order to examine the critical residues of four peptides for their localization and function, alanine mutants and deletion mutants were constructed as shown in tables 1 and 2. Numbering residues starting at the N-terminus of the peptide sequences. The cell tracker assay was performed for A549.tTA cells expressing GFP-fused peptides. The anti-proliferative effect of each GFP-fused peptide was analyzed and cellular localization of each GFP-fused peptide was examined. Results for mutations of all four peptides are summarized in tables 1 and 2.

In CAAX box containing proteins, the cys is prenylated to allow anchoring to a cell membrane (Hancock, J. F. et al., Embo J, 10:4033-4039 (1991)). Mutation of the cys 14 in #38 to ala blocked membrane localization and nuclear exclusion, and its anti-proliferative activity was completely abolished. Deletion of the N-terminal 8 residues of #38, while leaving the C-terminal CCIS intact, left localization intact, but anti-proliferative activity was significantly reduced. Deletion of the N-terminal 13 residues caused less significant membrane localization and nuclear exclusion and there was no significant anti-proliferative activity. As a comparison, the C-terminal 17 residues of the CAAX box peptide from CDC42 (CDC42C) was expressed as a GFP-fused peptide and analyzed for its anti-proliferative effect and cellular localization. This peptide was membrane-localized and nuclear excluded, but had no significant anti-proliferative activity. These results suggest that C14 is critical for activity and membrane localization, that the N-terminal 8 residues are also important for activity, and that the C-terminal 9 residues play a key role in its membrane localization. We then created point mutatants changing the N-terminal 8 residues). All #38 point mutatants in these residues except for K6A showed similar cellular localization as GFP-fusion #38. The K6A mutant had intense GFP localization in cytoplasmic granular structures and a reduced anti-proliferative effect. Interestingly, the anti-proliferative effect of S2A was enhanced and compatible with that of GFP-fused p21C.

To test the hypothesis that the localization and the anti-proliferative effect of #40, #41, and #88 are related to the leucine rich sequence motifs, alanine mutants were constructed at one or more sites and tested for both localization and function. Three mutants were constructed for #41: the single mutant L18A, the double mutant L18A/M16A, and the triple mutant L18A/M16A/L12A. Mutation of leu 18 resulted in a partial loss of the anti-proliferative effect but retained some localization to the membrane and perinuclear region. Additional mutation of met 16 to ala ablated all localization and reduced the anti-proliferative effect. The triple mutation resulted in no significant localization or anti-proliferative effect. All three mutated residues appear to be important for the growth inhibitory properties of #41. The anti-proliferative activity thus does not exactly coincide with the cellular localization.

For #40 as well, single and double ala mutants retain anti-proliferative activity without nuclear exclusion. Additional mutation of Phe 10 to ala ablated localization in membrane and cytoplasmic granule structures and significantly reduced the anti-proliferative effect. For #88, single and double mutants were localized to the cytoplasmic membrane structures and nuclear excluded, while the triple mutant was only slightly nuclear excluded. In addition cells expressing the double or triple mutant lost their anti-proliferative effect, however. Thus all three peptides have rough but not exact correlations between nuclear exclusion and their anti-proliferative activity. Multiple mutations in the leucine rich motif also resulted in loss of anti-proliferative effect of each peptide.

The Synthetic Peptide Analogue with the Fused Internalization Sequence Displaces GFP Fusion Peptides.

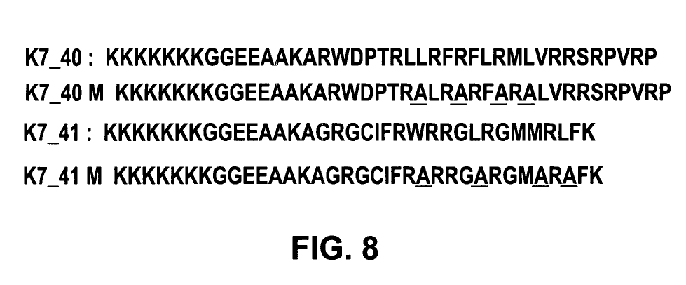

In order to examine whether it is necessary to express the peptides with the protein scaffold for their anti-proliferative effect, #40, #41 and their mutants with an altered leucine rich motif to four alanines (shown in FIG. 8) were synthesized. Since polybasic peptides can be efficiently internalized into cells (24-26), the peptides were fused with seven Lys (K7) via a linker (GGEEAAKA) at the N-terminus (K7#40, K7_{—}#40M, K7_{—}#41 and K7_{—}#41M).

The FITC-labeled K7_{—}#41 and K7_{—}#41M (K7_{—}#41-FITC and K7_{—}#41M_FITC) were also used for localization studies. A549 cells were plated on coverslips and incubated with 5 μM FITC-labled K7_{—}#41 (K7_{—}#41-FITC) or K7_{—}#41M (K7_{—}#41M-FITC) for 24 hours. Localization of the peptides was examined on a fluorescent microscope, Axiovert S100. The images were captured with a CCD camera, model C4742-95-12ER. K7_{—}#41_FITC and K7_{—}#41M_FITC were localized to not only the plasma membrane and cytoplasm, but also the nucleus, indicating that the peptides were effectively taken up by cells. Because of the nature of polybasic peptides (Vies, E. et al, J Biol Chem, 272:16010-16017 (1997); Futaki, S. et al., J Biol Chlem, 276:5836-5840 (2001)), the peptides were predominantly transported to the nucleus and nucleoli. No significant difference between the wild type and the mutant was seen. The FITC-labeled K6_{—}#40 and K6_{—}#40M also showed similar localization (data not shown).

GFP fused #38, #40, and #41 were concentrated at the plasma membrane and granular cytoplasmic structures. Especially significant GFP localization in the cell-cell contact area was seen in cells expressing GFP fusion #38 and #41. We examined whether synthetic analogues can displace cellular localization of GFP fusion peptides. A549 cells expressing GFP fusion #38 and #41 were incubated with 100 μM of K7_{—}#41, K7_{—}#41M and K7_{—}#40 for 5 hours and GFP localization was analyzed using a fluorescent microscope. A549 cells expressing GFP fusion #41 treated with K7_{—}#41 showed punctuated localization of GFP and loss of GFP localization in the cell-cell contact area. However these changes were not seen in cells treated with K7_{—}#41M nor K7_{—}#40. Surprisingly, similar changes were observed when A549.tTA cells expressing GFP fusion #38 were cultured with K7_{—}#41, but not K7_{—}#41M nor K7_{—}#40. These results suggest that K7_{—}#41 specifically altered the membrane localization of GFP fusion #41 and #38.

Synthetic Peptide Analogues Cause Apoptosis of A549 Cells.

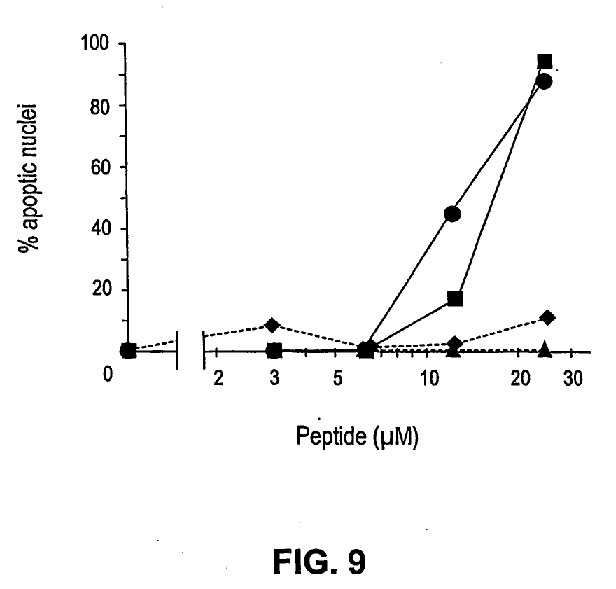

A549 cells were treated with K7_{—}#40, K7_{—}#40M, K7_{—}#41 and K7_{—}#41M for 24 hours and the number of nuclei and apoptotic nuclei in each well was counted as described in the materials and methods. Both K7_{—}#40 and K7_{—}#41 exhibited a dose-dependent decrease in the number of nuclei with a midpoint concentration of 10-15 μM, while K7_{—}#40M and K7_{—}#41M showed no significant reduction in nuclei number. Interestingly, a decrease in the number of nuclei observed in K7_{—}#40- and K7_{—}#41-treated A549 cells corresponded to an increase in apoptotic nuclei (shown in FIG. 9). K7_{—}#40_FITC and K7_{—}#41_FITC, but not K7_{—}#40M_FITC nor K7_{—}#41M_FITC, exhibited anti-proliferative effect with a midpoint concentration of 30-40 μM (data not shown).

Four anti-proliferative peptides emerged as hits from this screen. These peptides showed significant localization at the plasma membrane or granular cytoplasmic structures. One peptide, #38 contains a CAAX box motif and three others, #40, #41, and #88 have a leucine-rich motif. Synthetic analogs of two peptides exhibited dose-dependent apoptosis not seen in mutant peptides.

### Example 3

### Cellular Proteins that Interact with Anti-Proliferative Peptides were Identified Using Mass Spectrometry Based Shotgun Peptide Sequencing

Introduction

Cellular processes are tightly regulated by the activation and deactivation of series of proteins that constitute signaling pathways. An approach to discover novel elements of these pathways in the context of their functional biology will increase understanding of the pathways, of the phenotypes being studied, and may allow discovery of novel drug targets. Cellular screens of random peptide libraries have been used to discover novel peptides or proteins with specific cellular phenotypic effects in yeast (Caponigro et al., Proc Natl Acad Sci. 95:7508-13 (1998); Geyer et al., Proc Natl Acad Sci USA 96:8567-72 (1999); Norman et al., Science 285:591-5 (1999)) or in human cells following retroviral delivery of peptide libraries (Peelle et al., Chemistry & Biology 8:521-534 (2001); Xu et al., Nature Genetics 27:23-29 (2001); Kinsella et al., Journal of Biological Chemistry, 277:37512-8 (2002)). These screens do not require prior knowledge of protein interactions or signaling pathways involved in phenotypic changes, thus novel pathway members or interactions may be discovered. Peptide library members may bind cellular macromolecules as part of their phenotypic effect. Besides detailed analysis of the exact phenotypic effects, an important step towards understanding function involves determination of their cellular binding partners. Identification of these interacting partners may elicit hypotheses of mechanism, and may reveal potential drug targets for treating the disease.

Here we examine three antiproliferative peptides, discovered in a screen for antiproliferative peptides that are also associated with dose-dependent apotosis when added to the medium of A549 lung carcinoma cells, for cellular interactors by comparing LC/MS/MS-based shotgun peptide sequencing of tryptic digests of quadruplicate active peptide affinity extracts with inactive mutant peptide extracts. MALDI-TOF mass spectrometry of in-gel tryptic digests of difference gel bands, and Western blotting, also contributed to the discovery of interacting proteins. The three nuclear excluded peptides share a variant of a common leucine-rich nuclear exclusion motif, and bind to a common set of proteins involved in nucleocytoplasmic transports. The peptides also bind to proteins unique for each peptide, suggesting important differences.

Materials and Methods

Affinity Extractions and Gel Electrophoresis.

Affinity extractions and electrophoresis were carried out mainly as described (Gururaja et al.,. Journal of Proteome Research 1:253-261 (2002)). Briefly, cultured human A549 lung cancer cells (108 for each affinity extraction) were pelleted, washed twice in phosphate-buffered saline, and suspended in 5 mL of 4° C. 2% Triton X-100 containing a cocktail of protease inhibitors (Complete Tablets, Boehringer Mannheim, Germany), 20 mM tris buffer pH 7.2, 0.15M sodium chloride, and 1 mM ethylenediamine tetraacetic acid (EDTA). After homogenization, the cells were centrifuged at 14000×g for 20 min. at 4° C., the supernatant was cleared of proteins that may bind agarose beads by tumbling with 250 μl of a suspension of agarose beads (Pierce Chemical Co., Rockford, Ill.). The protein concentration as measured by a micro-BCA assay (Pierce Chemical Co., Rockford, Ill.) and was normalized to 5 mg/mL in all the affinity extractions. After centrifugation, 1 mL of the supernatant was added to 250 μL of a slurry of agarose beads covalently attached to streptavidin (Pierce Chemical Co., Rockford, Ill.) preincubated with biotinylated peptides (American Peptide Co., Sunnyvale Calif.) and washed three times in PBS. If free in solution the peptide concentrations would be ca. 50 μM in cell lysates. Quadruplicate extractions were carried out overnight at 4° C. Beads were then washed three times with 0.05M tris-0.15 M sodium chloride-0.1% triton X-100 followed by two washes with 0.05M tris-0.15 M sodium chloride-1 mM EDTA. Quadruplicate control extracts under identical conditions were obtained using biotinylated inactive tetra-alanine mutant peptides.

Washed beads were boiled in SDS-PAGE 2× sample buffer (Novex, San Diego, Calif.) containing 100 mM dithiothreitol (DTT). SDS-PAGE separation was on a Novex (San Diego, Calif.) 420% gradient Tris-glycine gel. Proteins electroblotted onto PVDF membranes (Novex, San Diego, Calif.) were probed with appropriate antibodies and developed using an ECL Plus enhanced chemiluminescence reagent kit followed by detection on ECL hyperfilm (Amersham Pharmacia Biotech, Piscataway, N.J.).

All antibodies used in Western blots were from BD Biosciences-Transduction Labs (Lexington, Ky.) except antibodies to PCNA and elongation factor tu (Santa Cruz Biotechnology, Santa Cruz Calif.).

Mass Spectrometry Analysis of Affinity Extracts.

Microcapillary LC/MS/MS analysis was carried out generally as described (Gururaja et al., Journal of Proteome Research 1:253-261 (2002)). Briefly, 5 μL of lys C/trypsin digests of streptavidin-bead affinity extracts were desalted offline using a C18 guard column (Vydac, Hesperia, Calif.), and injected onto a fused silica microcapillary HPLC column (15 cm long, 75 um i.d.) packed with Nucleosil C18 (100 Å pore size, 5 mm particles). The capillary column was connected to a 15 mm PicoTip (New Objective Inc., Woburn, Mass.) as the electrospray tip through a stainless steel zero dead volume union where the electrospray voltage was applied. HPLC solutions were 5% acetonitrile, 0.1% formic acid (buffer A) and 80% acetonitrile, 0.1% formic acid (buffer B). The HPLC gradient was a 20 min gradient from 0 to 10% buffer B, a 60 min gradient from 10 to 30% buffer B, a 10 min gradient from 30 to 50% buffer B, a 10 min gradient from 50 to 80% buffer B, and hold at 80% buffer B for 10 min. Precursor ions were scanned from 350-1800 m/z in full-scan mode. The HPLC was an Ultimate capillary HPLC (LC Packings, San Francisco, Calif.). MS/MS used a ThermoFinnigan LCQ ion trap mass spectrometer. Some samples were run on a Micromass (Beverly Mass.) QTOF-1 mass spectrometer. In-gel digest peptide extracts were analyzed on a Bruker Reflex m time-of-flight mass spectrometer at both Rigel and the Univ. of Alberta, and a Bruker Anchor Chip using dihydroxybenzoic acid as a matrix (Rigel); some peptides were also fragmented using a PE-Sciex API-QSTAR pulsar at the Univ. of Alberta.

Database Searching and Medusa Analysis.

Collected MS/MS data were analyzed using TurboSequest software (ThermoFinnigan, San Jose, Calif.) against a human protein sequence database derived from the NCBI nonredundant database. Human immunodeficiency virus protein sequences were first removed from the human protein database by a database tool in the Xcalibur software (ThermoFinnigan, San Jose, Calif.). SEQUEST scores (Eng et al., J. Am. Soc. Mass Spectrom. 5:976-989 (1994)) were evaluated by the criteria of Washburn et al. (Washburn et al., Nature Biotechnol. 19:242-247 (2001)]) which includes peptides with a delta Cn of greater than 0.1, and Xcorr values greater than 1.9, 2.2 and 3.75 for +1, +2 and +3 ions, respectively. SEQUEST results were summarized and stored in an Oracle 8i database called Medusa. Peptide masses obtained from MALDI-TOF experiments were analyzed using Mascot (Perkins et al., Electrophoresis 20:3551-67 (1999)) or ProFound (Zhang et al., Analytical Chemistry 72:2482-89 (2000)).

Fluorescence Microscopy Examination of A549 Cells.

A549 tumor cells (American Type Culture Collection, Fairfax Va.) were plated at 2000 cells per well in 96 well plates with F12k media supplemented with 10% fetal bovine serum and 1% penicillin/streptomycin and allowed to grow for 24 hours. Peptide dissolved in media was added to the cells to a final concentration of 0.8 to 100 uM in triplicate. Following incubation with peptide for 24 or 48 hours, cells were fixed with 3.7% formaldehyde for 30 minutes, rinsed in PBS and stained with DAPI (Molecular Probes, OR) to label nuclear DNA. Digital images of the labeled nuclei in each well were taken using a Zeiss Axiovert microscope, UV filter set and Photometrics camera. Images were analyzed using the Cellomics (Pittsburgh, Pa.) Image Pro software package to count the number of nuclei, measure the DNA staining intensity within each nucleus and thus assign a cell cycle stage to that cell, and calculate the percentage of nuclei exhibiting the fragmentation characteristic of late apoptosis.

Results

Cellular Localization of Individual Peptides.

Since the peptides active in a screen for antiproliferative activity were fused to GFP, the localization in A549 cells of these GFP fusions was examined. Confocal fluorescence microscopy was used to examine peptide localization; all but a control peptide were nuclear excluded. Peptide 41 appeared in different locations in different individual cells; in a coarse perinuclear structure in some cells, between dividing cells, or near the plasma membrane. Peptide 40 appeared in a coarse granular cytoplasmic structure, and peptide 35 was concentrated in a peri-nuclear structure. The control peptide was not nuclear excluded. Peptides 35 and 41 were completely nuclear excluded, and peptide 40 was partially excluded. Besides appearance in granular cytoplasmic structures, in different individual cells, peptide 41 was concentrated in a dense area next to the nucleus, was localized to the cell-cell contact area, or was concentrated in the plasma membrane. Peptide 40 was concentrated in coarse cytoplasmic structures. Peptide 35 appeared cytoplasmic but was concentrated in a coarse granular structure. Besides being nuclear excluded, each peptide's localization was unique. A destabilized GFP control was distributed throughout the cells.

Peptide Sequences may Explain Peptide Nuclear Exclusion.

Since all of the peptides are nuclear excluded, their sequences were aligned with those of nuclear excluded proteins with leucine-rich nuclear exclusion motifs (Table 3). Such proteins, such as HIV-1 Rev or HTLV-1 Rex, have a leucine-rich motif and are recognized by a complex including exportin-1 for transport out of the nucleus (Yoneda et al., Cell Struct. Fct. 24:425-33 (1999); Henderson et al., Exp. Cell Res. 256:213-224 (2000); Chook et al., Current Opin. Structural Biol. 11:703-715 (2001)). The leucine-rich motifs are characterized by three leucines separated by 2-3 other residues, and a fourth leucine separated by a single residue. The leucines are sometimes replaced by other hydrophobic residues such as phe or ile. If other hydrophobic residues such as met and trp are allowed to replace some leucines, peptides 35, 40 and 41 have sequences that fit this more general motif.

TABLE 3

SEQUENCE ALIGNMENTS OF THREE ANTIPROLIFERATIVE PEPTIDES
WITH NUCLEAR EXCLUSION MOTIFS.

| | | |
|---|---|---|
| peptide | sequence* | comment |

| | | |
|---|---|---|
| 35 | RLRRICSGILL IRRI LG IFV | |
| 40 | RWDPTRLLR FRF LR MLVRRSRPVR | |
| 41 | GRGCIFRWRRGLRGM MR LFK | |
| HIV-1 Rev | LPP LER LT LD | leu rich motif |
| HTLV1 Rex | LSAQLYSS LS LD | leu rich motif |
| PKI | LALKLAG LD IN | leu rich but I substutes for L |
| HDM-2 | LSLSFDES LA LC | leu rich but F substitutes for L |
| general motif | X X X X | X is a hydrophobic residue |

*THE SEQUENCES SHOWN ARE THOSE OF THE RANDOM PEPTIDE LIBRARY MEMBER; ALL WERE FUSED TO THE GFP C-TERMINUS VIA A SPACER SEQUENCE -EEAAXA-.

Synthetic Peptide Analogs with Fused Internalization Domains Cause Apoptosis of A549 Cells.

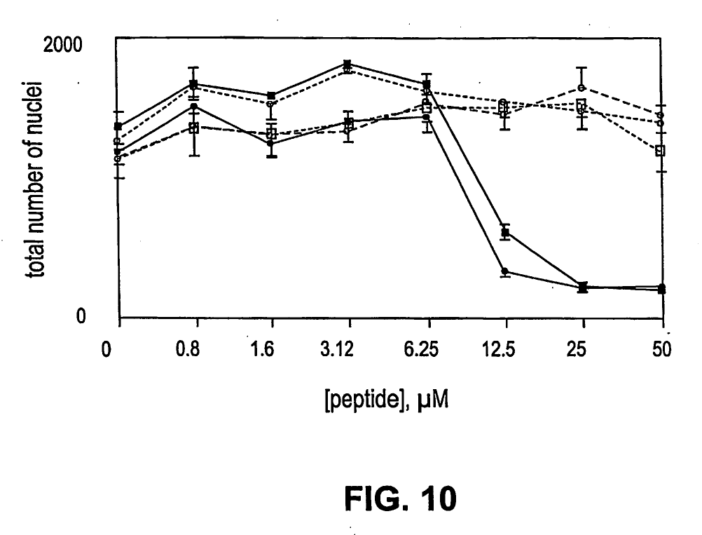

Polybasic peptides can efficiently internalize fused macromolecules into cells (Vives et al., J Biol Chem 272:16010-7 (1997); Futaki et al., J Biol Chem 276:5836-40 (2001); Lindsay, M., Curr Opin Pharmacol 2:587-94 (2002)). The effects of N-terminal lys7-tagged peptides 40 and 41 on A549 cells were examined using fluorescence microscopy (Blake, R., Curr Opin Pharmacol. 1:533-9 (2001)). FIG. 10 shows the results of the peptide dose-dependence of the number of cell nuclei remaining in a 96 well plate, originally seeded with 2000 cells, after 48 hours exposure to peptides added to the cell culture medium. Tetra-alanine mutants of both peptides showed no dose-dependent effect up to 50 mM levels. Both peptides 40 and 41 exhibited a dose-dependent decrease in the number of cell nuclei observed, with a midpoint concentration of ca. 10 mM.

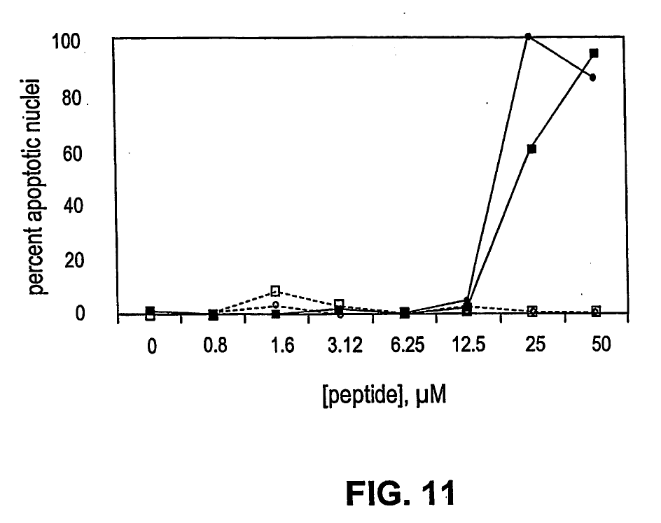

FIG. 11 shows the dose-dependence of apoptosis for peptides 40 and 41 after 48 hours exposure to A549 cells. The control alanine-mutant peptides had no observable effect, while both lys7-tagged peptides were associated with a dose-dependent increase in apoptotic nuclei with midpoints in the 10-20 mM range. The majority of the remaining cells for 25 and 50 mM doses exhibited the severely fragmented nuclear morphology characteristic of late apoptosis.

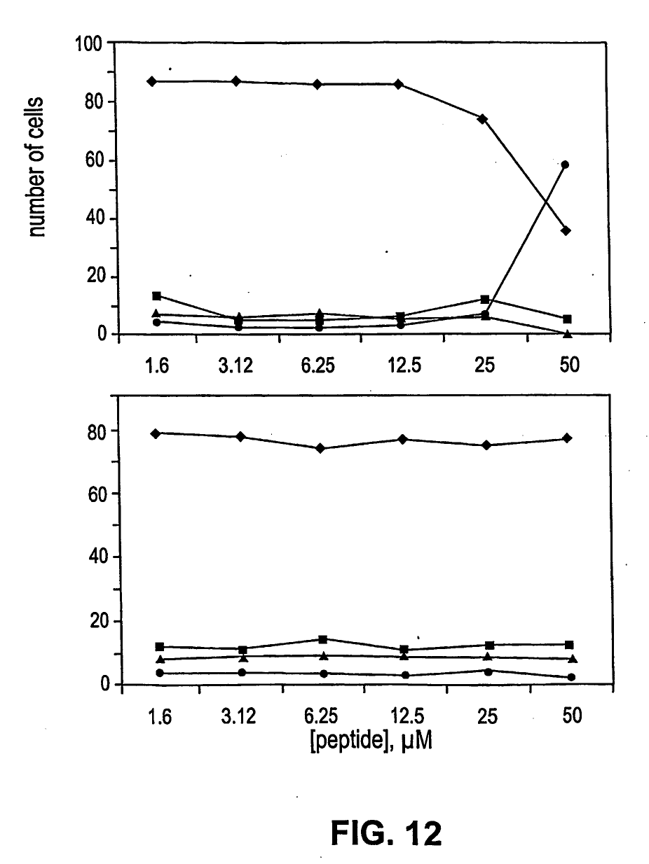

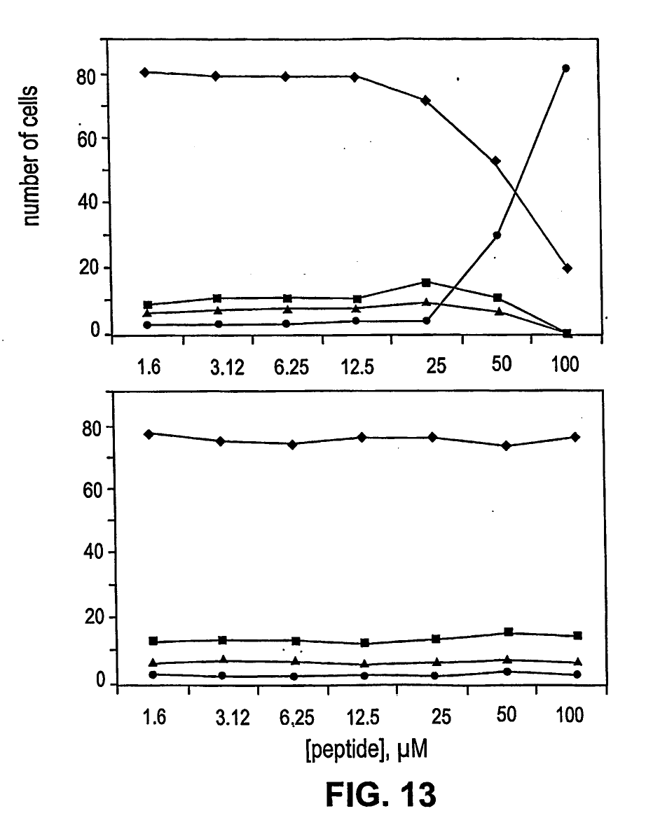

FIG. 12 shows the dose-dependence of the cell cycle stage after 48 hours exposure of asynchronously growing A549 cells to peptide 41. These are cells remaining after 48 hours which have not undergone apoptosis. No change in the cell cycle was observed for the lys7-tagged control alanine mutant peptide at any dose. At 25 and 50 mM levels of peptide 41, G1 phase cells were depleted and the percent of M phase cells increased significantly. A similar effect was observed for lys7-tagged peptide 40 (FIG. 13). The alanine-mutant peptide caused no change in the cell cycle distribution of the A549 cells, even at high doses. Active peptide 40 caused a depletion of G1 cells, a complete loss of S and G2 cells at 100 mM levels, and an increase in M phase cells. Lys7-tagged peptides 40 and 41, but not the alanine-mutant control peptides, thus can cause a dose-dependent cell cycle arrest or apoptosis in A549 cells.

In-Gel Tryptic Digests of Difference 1D Gel Bands and MALDI-TOF Mass Spectrometry Identify Binding Partners of 3 Peptides.

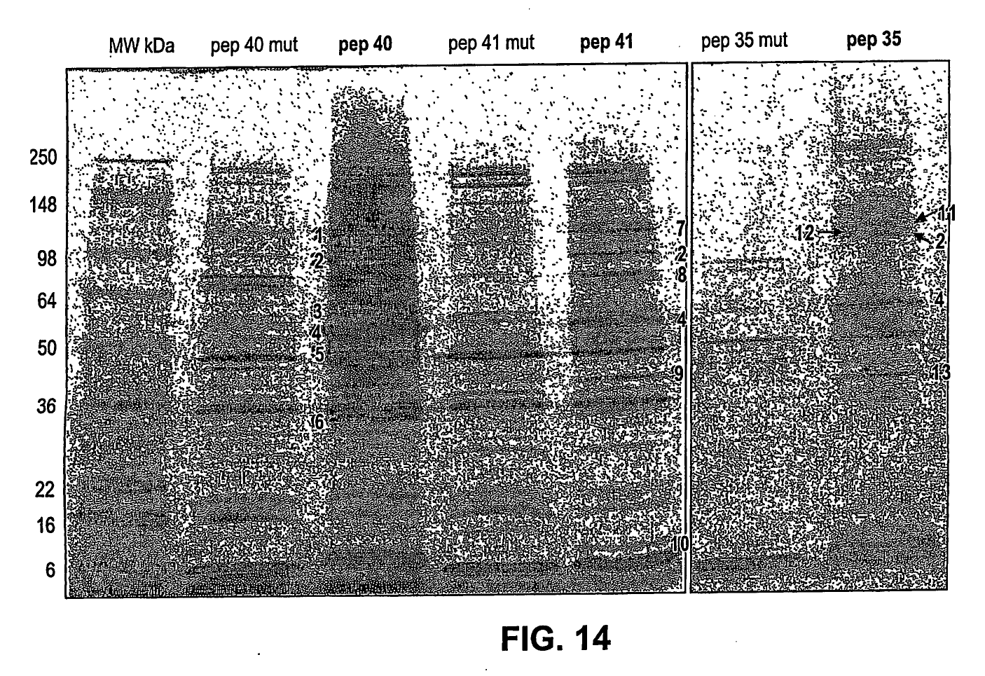

To further examine the action of these peptides in cells, peptides 35, 40 and 41 were synthesized with the N-terminal sequence biotin-GMDELYKEEAAKA-. The residues MDELYK were from the C-terminus of GFP; the residues EEAAKA were spacer residues between the GFP beta-can structure and the peptide sequence selected in the functional screen. The GFP beta can was thus replaced with a biotin. Inactive alanine mutant peptides, with all four of the bold residues shown in Table 3 mutated to alanine, were used as controls. A549 cell affinity extracts using the active peptide sequences were compared to control extracts using 1-dimensional silver stained gels. FIG. 14 shows the results of this comparison for peptides 41, 40 and 35. Difference bands were identified using MALDI-TOF mass spectrometry and matching the masses of the in-gel digest tryptic peptides to predicted peptide masses for individual proteins using the programs Mascot (Perkins et al., Electrophoresis 20:3551-67 (1999)) or Profound (Zhang et al., Analytical Chemistry 72:2482-89 (2000)). The resulting identifications, with the number of matching tryptic peptides, are included in the middle columns of Table 4 for peptide 41. Each identified protein had a predicted molecular mass within 10% of the observed mass. Seven interacting proteins, including three subunits of importin-beta, beta-tubulin, BiP, annexin II and calpactin-1/S100, were identified for peptide 41. Each was identified by 10-20 matching tryptic peptides.

TABLE 4
Combined identification of protein interaction partners of peptide 41.

| | # | 1D gel, MALDI- | gel | # | |
|---|---|---|---|---|---|
| LC/MS/MS/Medusa | peptides | TOF MS | band | peptide | Western blot |
| β tubulin 2 | 3, 3* | β tubulin | 4 | 13 | |
| β tubulin 5 | 3, 4 | | | | |
| α tubulin | 6, 6 | | | | |
| myosin heavy chain | 2, 3 | | | | importin α |
| α, β □□ γ-actin | 2, 2 | | | | nucleoporin p62 |
| | | | | | exportin-1 |
| (importin β2) | 0, 1 | importin β1 | 2 | 16 | importin β |
| | | importin β3 | 7 | 14 | Ran |
| | | importin β7 | 7 | 13 | |
| annexin II | 2, 2 | annexin II | 9 | 13 | annexin II |
| calpactin/S-100 | 0, 3 | calpactin I/S100 | 10 | 10 | calpactin I/S100 |
| elongation factor tu | 2, 3 | | 3 | | elongation factor tu |
| elongation factor 1α-1 | 4, 4 | | | | |
| glycyl-tRNA synthetase | 3, 3 | | | | |
| hnRNP-U | 1, 2 | | | | |
| hnRNP-F | 1, 1 | | | | hnRNP-F |
| protein phosphatase 1G/2Cγ | 1, 2 | | | | |
| | | | | | HSP 70 |
| DNA-binding protein A | 2, 2 | | | | PCNA |
| | | BiP/GRP-78 | 8 | 20 | BiP/GRP-78 |

*The number of good peptides identifying a protein, and total peptides, are listed in sequence.

For peptide 40 (Table 5) six binding partners were identified, including importin-beta subunits 1 and 3, beta-tubulin, elongation factor tu, a Kruppel-type zinc finger, and an ATP/ADP carrier protein. All were identified by at least 11 peptides except importin-beta 3, which was identified by 8 peptides. All identified proteins were within 10% of the observed mass except for the zinc finger protein, which was within 22%.

TABLE 5
Combined identification of protein interaction partners of peptide 40.

| | | 1D gel, MALDI-TOF | gel | # | |
|---|---|---|---|---|---|
| LC/MS/MS | # pep | MS | band | peptides | Western blot |
| β tubulin | 7, 8 | β tubulin | 4 | 13 | |
| α tubulin | 9, 9 | | | | |
| α-actin | 2, 3 | | | | |
| | | importin β1 | 2 | 16 | importin β |
| | | importin β3 | 1 | 8 | exportin-1 |
| | | | | | nucleoporin p62 |
| | | Kruppel-type Zn finger | 3 | 11 | importin α |
| | | | | | Ran |
| SF2p32 splicing factor | 6, 7 | | | | |
| hnRNP-M | 2, 3 | | | | hnRNP-F |
| elongation factor tu | 5, 6 | elongation factor tu | 5 | 12 | elongation factor tu |
| elongation factor 1α-1 | 3, 6 | | | | |
| ribonuclease inhibitor | 3, 3 | | | | |
| DNA ligase-like protein | 1, 1 | | | | |
| PCNA | 1, 1 | | | | PCNA |
| nucleobindin 2 | 2, 2 | | | | calpactin |
| reticulocalbin 1 | 5, 5 | | | | |
| reticulocalbin 2 | 1, 1 | | | | |
| calumenin | 4, 5 | | | | |
| paraoxonase 2 | 2, 2 | ATP/ADP carrier protein | 6 | 15 | |
| ATP synthase α,β subunits | 2, 2-β | | | | |
| | 2, 2-α | | | | |
| pyrroline-5-carboxylate | 3, 3 | | | | |
| reductase | | | | | |

For peptide 35 (Table 6), five interacting partners were identified. These included importin-beta 1, beta-tubulin, actin, restin, and KIAA0052. Thus these three functional peptides appear to share interactions with a number of proteins, as well as bind to proteins unique for each peptide.

TABLE 6
Combined identification of protein interaction partners of peptide 35.

| | | 1D gel, | | | |
|---|---|---|---|---|---|
| | # | MALDI | gel | # | |
| LC/MS/MS | peptides | TOF MS | band | peptides | Western Blot |
| β tubulin | 4, 4 | β tubulin | 4 | 13* | |
| α tubulin | 4, 6 | restin | 12 | 12 | |
| | | | | | importin α |
| | | importin β1 | 2 | 16 | importin β |
| | | | | | exportin-1 |
| | | | | | nucleoporin |
| | | | | | p62 |
| | | | | | Ran |
| | | | | | RCC1 |
| | | KIAA0052 | 11 | 10 | |
| (DNA-directed | 0, 1 | | | | |
| DNA pol II) | | | | | |
| DNA-directed | 1, 1 | | | | PCNA |
| RNA | | | | | |
| Polymerase IID | | | | | |

LC/MS/MS-Based Shotgun Peptide Sequencing and Western Blotting Identify Additional Interacting Proteins.

To find additional interacting proteins, quadruplicate affinity extracts using each biotinylated peptide were separately digested with lys-C endoprotease and then with trypsin. Quadruplicate extracts using the control biotinylated inactive peptides, each of which had the four nuclear exclusion motif residues (Table 3) mutated to alanine, were digested in the same fashion. The entire affinity extracts were then chromatographed on a microcapillary C18 reversed phase hplc column, the mass of each peptide was measured, and it was then fragmented in an ion trap mass spectrometer. This LC/MS/MS data was then compared to data from the control affinity extracts using the Oracle database MEDUSA (Gururaja et al., Journal of Proteome Research 1:253-261 (2002)). Proteins unique to the active peptide affinity extracts, with at least one good sequenced peptide using published criteria (Washburn et al., Nature Biotechnol. 19:242-247 (2001); Gygi et al., J. Proteome Res. 1:47 (2002)), and present in at least two of four extracts, were identified. The individual tryptic peptides used for the identifications were independently BLASTed to check for ambiguity in identifications. The number of good peptides, and total peptides, used in the identifications are listed in Tables 4-6.

For peptide 41, this mass spectrometry data revealed the presence of two types of beta-tubulin, alpha-tubulin, actin, as well as myosin. Importin-beta 2, annexin II and calpactin were also found. Two elongation factors, two hnRNP proteins, a protein phosphatase, a t-RNA synthetase and a DNA-binding protein were discovered. For peptide 40, alpha and beta-tubulin and alpha-actin were present, as were a splicing factor and hnRNP protein, two elongation factors, two proteins associated with DNA replication, proliferating cell nuclear antigen (PCNA) and a DNA ligase-like protein, two reticulocalbins, calumenin, nucleobindin, two unknown proteins and several enzymes. For peptide 35, fewer proteins were reproducibly present in the affinity extracts. These included alpha- and beta-tubulin and DNA-directed RNA polymerase RD.

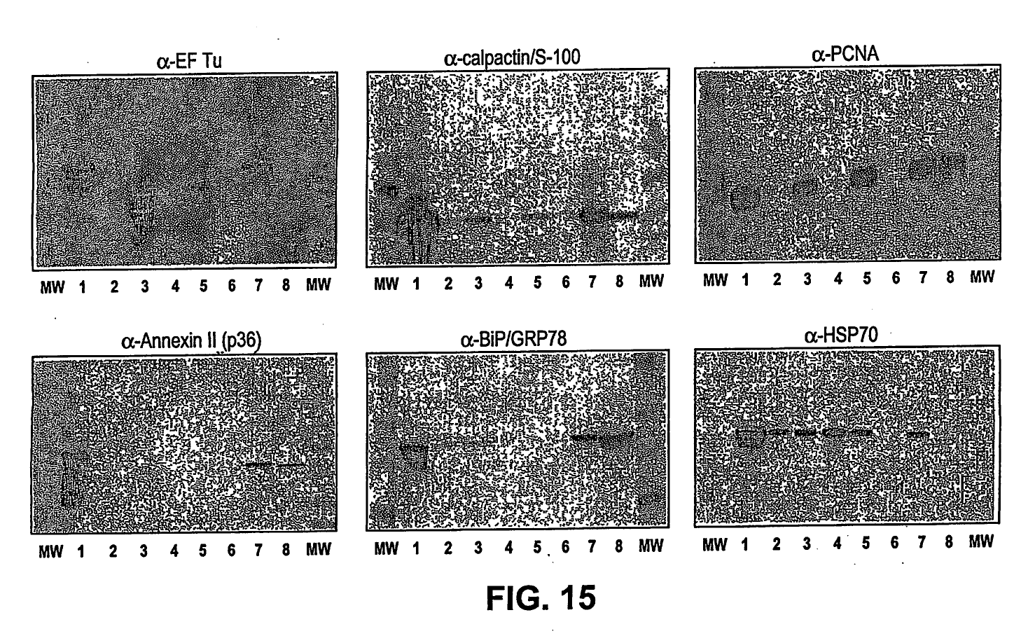

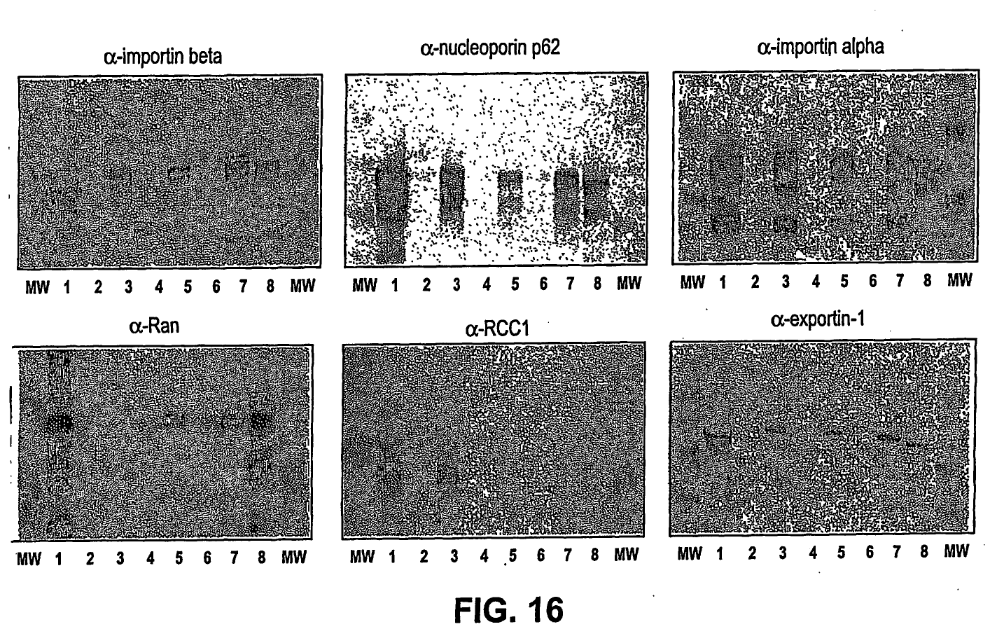

To confirm the presence of a number of these proteins, Western blotting was done on the affinity extracts when antibodies were available. Blots of whole cell lysates were included, as well as positive control proteins when available. Blotting was not done for tubulin, actin and myosin since the identifications from LC/MS/MS data involved multiple sequenced peptides. FIG. 15 shows the results of Western blots for proteins listed in Tables 4-6. The presence of PCNA was confirmed in affinity extracts of all three active peptides but not in the extracts using the mutant peptides. Calpactin/S-100 was present in two extracts but not in controls, and more was present in a third extract than in the control. Annexin II, BiP and elongation factor tu were differentially present in the peptide 41 extract, but were not observable in the other extracts. Elongation factor tu and heat shock protein 70 were differentially present in the peptide 41 extract, and a lower band was differentially present in the peptide 40 extract. Due to the presence of importin beta subunits indicated by combined mass spectrometry data, antibodies binding to other nuclear pore complex proteins were also tested in Western blots. FIG. 16 shows data from Western blots for nuclear pore complex proteins. Importin-beta, importin-alpha, nucleoporin p62, the GTPase ran, and exporting are present in all three peptide affinity extracts but not in control extracts. Up to three bands were visible for importin-beta, which may reflect the presence of more than one subunit with differing masses, consistent with the MALDI-TOF mass spectrometry data for peptides 40 and 41. RCC1, a nuclear GDP-GTP exchange factor promoting the GTP-bound form of ran GTPAse, was present in the peptide 35 extract but at or below the limit of detection in extracts of the other peptides. Importin alpha and nucleoporin may be partially degraded in these preparations as indicated by the presence of multiple bands.

A summary of identified interacting proteins for each peptide, obtained from the three different methods, is contained in Table 7. All of the peptides differentially bind cytoskeletal proteins. These include tubulin, actin and myosin, and the microtubule associated protein restin (Delabie et al., Leuk Lymphoma 12:21-26 (1993)), which may regulate microtubule stability (Choi et al., Curr Biol 10:861-864 (2000)) and is involved in the binding of endosomes to microtubules. Peptide 40 binds significantly more tubulin than peptides 35 or 41, suggesting that polymerized tubulin may interact with this peptide. Other sets of interactors are discussed below.

TABLE 7
Sets of protein interactors of three functional peptides identified
by affinity mass spectrometry.

| | | nucleocytoplasmic | protein synthesis; | ER or Golgi | DNA, replication |
|---|---|---|---|---|---|
| peptide | cytoskeletal | transport | pre-mRNA splicing | proteins | related |
| 40 | tubulin | importin β | EF-tu | reticulocalbin 1, 2 | DNA ligase-like |
| | actin | exportin 1 | EIF1α1, 2 | calumenin | PCNA |
| | | nucleoporin p62 | hnRNP M | nucleobindin 2 | Kruppel Zn finger |
| | | importin α | hnRNP F | | calpactin 1 |
| | | ran | SF2p32 pre-mRNA | | |
| | | | splicing factor | | |
| | | | rnase inhibitor | | |
| 41 | tubulin | same as above | EF-tu | | PCNA |
| | myosin | | EIF 1α1, 2, β1-like | | DNA-binding |
| | | | | | protein A |
| | actin | | hnRNP-U, F | | calpactin |
| | | | protein phosphatase | | annexin II |
| | | | 1G | | |
| 35 | tubulin | same as above, | DNA-directed RNA | | PCNA |
| | | | polymerase IID | | |
| | actin | RCC1 | | | DNA-directed DNA |
| | | | | | polymerase ∈ |
| | restin | | | | calpactin |

We have examined the interacting partners of three peptides which are antiproliferative when produced within A549 cells. Two were N-terminally fused to a lys7 sequence and, when added outside cells, produced a dose-dependent apoptosis in most cells and altered the cell cycle of remaining cells. This is a specific effect linked to a nuclear exclusion motif in these peptides since mutations of four motif residues to alanine abolished this activity. Although all three peptides are nuclear-excluded when fused to GFP, their cellular localization can be complex. Peptide 35 is localized mainly in a perinuclear structure, and peptide 40 resides mainly in a cytoplasimc granular structure and in the cytoplasm. Peptide 41 can localize in different cells to the plasma membrane, nuclear membrane, a perinuclear structure, and to a granular structure in the cytoplasm, consistent with interactions with macromolecules in different cellular locations. Peptides 35, 40 and 41 bind importin beta, which associates with annulate lamellae in the cytoplasm (Cordes et al., Exp Cell Res 237:419-33 (1997)), which could partly explain the cytoplasmic localization of one or more of these peptides.

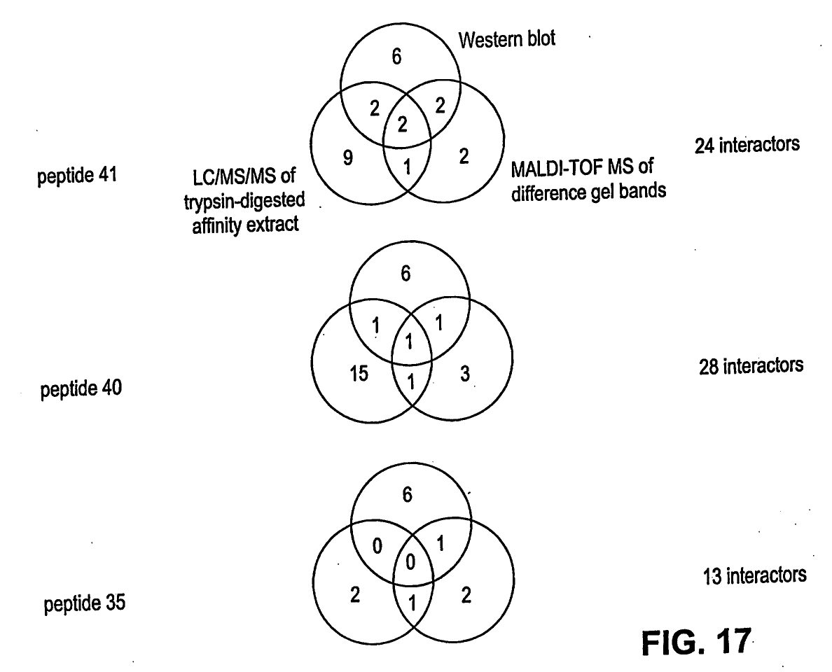

We have used differential affinity extractions, comparing biotinylated active peptides with tetra-alanine mutants, to identify interacting proteins that may be involved in the function of these peptides. Combining MALDI-TOF MS, capillary LC/MS/MS, and Western blotting, a total of 13, 28 and 24 interacting proteins were obtained for peptides 35, 40, and 41. FIG. 17 shows Venn diagrams of the results of these different methods of identifying interacting proteins. Western blotting confirmation of all proteins was not attempted, thus more proteins could be identified by this method. Each method contributes unique interactors; proteins common to more than one method help validate the methodologies. A comprehensive examination of cellular interactors should include all three methods. Additional interacting proteins may be missed if the four mutated residues in the control peptides are not important for their binding to the peptide; these differential extracts may at least partly select proteins binding the leucine rich NES motif. These peptides bind common sets of proteins, but also unique proteins (Table 7). Peptide 35 may weakly bind more proteins also extracted by peptides 40 and 41, as a number of these proteins (elongation factor tu, reticulocalbin 1 and 2, calumenin, DNA binding protein A, hnRNP-F, splicing factor 2 p32, myosin heavy chain, actin, importin beta 1, and ATP synthase) were present in one of four LC/MS/MS-examined affinity extracts but were absent from the quadruplicate controls.

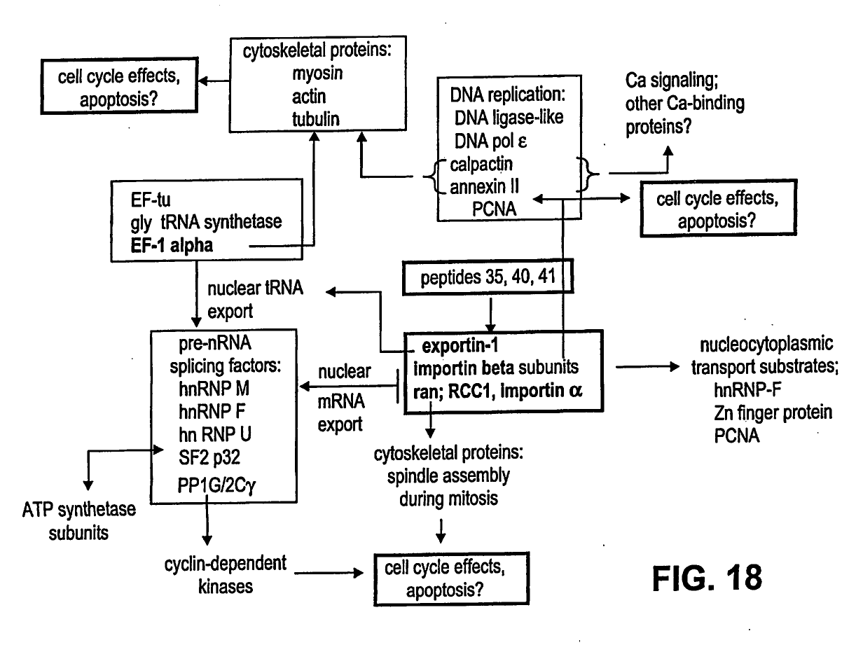

The individual peptides bind common sets of proteins, suggesting similarities in their mechanism of action, and unique proteins, suggesting some differences in their action. They also bind multiple functional classes of proteins, and may thus be present in more than one complex. The range of intensities of the difference silver stained bands are consistent with formation of different complexes present at different levels. Individual peptides also have unique interacting proteins. There may thus be more than one mechanism for the antiproliferative activity of the peptides. Additional sets of bound proteins are functionally linked to the nucleocytoplasmic transport system and to each other (FIG. 18). The extracted proteins may thus represent a snapshot of proteins linked both to the nucleocytoplasmic transport system and to antiproliferative or pro-apoptotic function in these cells.

One set of extracted proteins consists of nucleocytoplasmic transport proteins including exportin-1, several different subunits of importin beta, importin alpha, nucleoporin p62, the GTPase ran, and for peptide 35, the ran guanine nucleotide exchange factor RCC1. The isolation of these proteins is consistent with the observed nuclear exclusion of these peptides and the presence of a motif very similar to the leucine rich nuclear exclusion motif, validating the identification of interacting proteins by this approach. Peptide-mediated antiproliferative activity or apoptosis could thus involve disregulation of the nucleocytoplasmic transport system. Aberrant nucleocytoplasmic transport of transcription factors (Linden et al., Braz J Med Biol Res 1999 July; 32(7):813-20 (1999)) has been linked to apoptosis in retinal cells. Exportin-1 is involved in the nuclear export of proteins with the leucine rich NES motif (Stade et al., Cell 90:1041-50 (1997); Pemberton et al., Curr. Opin. Cell Biol. 10:392-9 (1998)) and interacts with and requires the GTPase ran (Moroianu, J., J Cell Biochem 75:76-83 (1999)), also isolated here. Nucleoporin p62, isolated with all three peptides, binds the importin alpha and beta heterodimer, along with ran, during nuclear import (Percipalle et al., J Mol Biol 266:722-32 (1997); Ben-Efraim et al., J Cell Biol 152:411-7 (2001)). Peptides 40 and 41 extract multiple subunits of importin beta. Complexes involving importin beta are not currently thought to contain multiple different importin beta subunits, thus these peptides could bind independently to each of several different importin beta subunits. Peptide 41 binds hnRNP-F and importin beta-2, a known importer-substrate pair (Chook et al., Current Opin. Structural Biol. 11:703-715 (2001)). Peptide 40 binds a Kruppel type zinc finger; members of this family are nuclear localized due to basic residues within the zinc finger common to all family members (Pandya et al., J Biol Chem 277:16304-12 (2002)). These peptides thus also affinity extract importer-substrate pairs. Besides containing a sequence similar to the leucine rich nuclear export motif, peptides 35, 40 and 41 are highly basic, containing 5, 9 and 7 basic residues respectively. None of the peptides contain a classical or bipartite nuclear localization motif thought to allow binding to importin alpha (Conti et al., Cell 94:193-204 (1998)). However the basic residue compositions of 25%, 38% and 35% are in the range of those of partially (30%) and fully nuclear-localized peptides (36%) derived from the same GFP C-terminally fused random 20mer peptide library (Peelle et al., Chemistry & Biology 8:521-534 (2001)), and these peptides do bind importins alpha and beta. They may act by binding both the nuclear export and import apparatus and compromising nucleocytoplasmic transport.

Since all three peptides are nuclear excluded, they could sequester nuclear proteins critical for cell proliferation outside the nucleus. One such protein is PCNA, which is critical for initiation of DNA synthesis and for DNA replication and repair (Niura, M., J Radiat Res (Tokyo) 40:1-12 (1999)). Antisense-oligonucleotides to PCNA block cell proliferation (Jaskulski et al., Science 240:1544-6 (1988); Speir et al., Circulation 86:538-47 (1992)) and prevent entry of G1 cells into S phase (Liu et al, Biochemistry 28:2967-74 (1989)). The ran mutants Q69L and T24N block PCNA accumulation in the nucleus and block DNA replication (Hughes et al., J Cell Sci 111:3017-26 (1998)). A second candidate is the GTPase ran, which is critical for mitosis, regeneration of the nuclear envelope and nuclear assembly, nucleocytoplasmic transport of a variety of macromolecules, DNA synthesis and replication, RNA processing and export (basso et al., EMBO J. 13:5732-44 (1994); Dasso, M., Prog Cell Cycle Res 1:163-72 (1995); Moore, J., Bioessays 23:77-85 (2001)). A third candidate is the nuclear guanine nucleotide exchange factor RCC1, which is essential for nucleocytoplasmic transport (Izaurralde et al, EMBO J. 16:6535-47 (1997)). Loss of RCC1 induces G1 arrest and defects in nuclear transport (Seki et al., T., J Biochem (Tokyo) 120:207-14 (1996)) and disruption of nuclear assembly and DNA replication (Dasso et al., EMBO J. 13:5732-44 (1994)).

A second set of extracted proteins includes protein synthesis-related proteins: elongation factor tu, elongation initiation factors 1 alpha 1 and 2, and glycine tRNA synthetase. Besides binding aminoacyl-tRNA and ribosomes, elongation factor 1 alpha binds actin filaments and microtubules (Murray et al., J Cell Biol 135:1309-21 (1996)) in a calcium/calmodulin regulated fashion (Kurasawa et al., J Biochem (Tokyo) 119:791-8 (1996)) and severs stable microtubules during the cell cycle (Shiina et al., Science 266:282-5 (1994)).

Peptides 41 and 40 also bind a third set of proteins, a total of 5 different pre-mRNA splicing factors. These include the hnRNPs M (Datar et al., Nucleic Acids Res 21:439-46 (1993)), F and U (Kiledjian et al., EMBO J 11:2655-64 (1992)), the splicing factor 2 associated protein p32, and protein phosphatase 1G/2Cγ (Murray et al., Genes Dev 13:87-97 (1999)). hnRNP F is a pre-mRNA splicing factor (Yoshida et al., FEBS Lett 457:251-4 (1999a)) imported into the nucleus using transportin/importin beta 2 (Pemberton et al., Curr. Opin. Cell Biol. 10:392-9 (1998), Siomi et al., J Cell Biol 138:1181-92 (1997)), which binds RNA polymerase II (Yoshida et al., Genes Cells 4:707-19 (1999b)). Importin beta and RNA polymerase II are both present in the peptide 35 extract. Protein phosphatase 1G/2Cγ is physically and functionally associated with pre-mRNA splicing (Murray et al., Genes Dev 13:87-97 (1999)). Type 2C protein phosphatases also dephosphprylate cyclin-dependent kinases (Cheng et al., Genes Dev 13:2946-57 (1999)). Protein serine/threonine phosphatase 1G/2Cγ expression blocks DNA synthesis and results in accumulation of cells in early G1 and S phases of the cell cycle (Guthridge et al., Mol. Cell. Biol. 17:5485-5498 (1997)). The splicing factor 2 associated protein p32 may regulate RNA splicing by blocking splicing factor 2-RNA binding and phosphorylation (Petersen-Mahrt et al., EMBO J 18:1014-24 (1999)). It is also found in the mitochondrial matrix, where it is hypothesized to play a role in oxidative phosphorylation (Muta et al., J Biol Chem 272:24363-70 (1997)). Interaction of this protein with oxidative phosphorylation proteins could explain the presence of ATP synthetase subunits a and 0 in the peptide 40 affinity extract.

The second and third sets of extracted proteins may be related. Nuclear export of mRNA is coupled to pre-mRNA splicing (Reed et al., Cell 108:523-31 (2002)), and mRNA splicing, the transcriptional apparatus, and transcriptional elongation are coupled in eukaryotic cells (Yoshida et al., Genes Cells 4:707-19 (1999b), Misteli et al., Mol Cell 3:697-705 (1999)) through an RNA polymerase-splicing protein complex (Fong et al, Nature 414:929-33 (2001); Robert et al., J Biol Chem 277:9302-6 (2002)). Nuclear tRNA export depends on exportin, elongation factor 1 alpha, and tRNA aminoacylation, coupling the protein translation, nuclear tRNA processing and transport machineries (Grosshan et al., J Struct Biol 129:288-94 (2000)). Ran and importin beta can function as nuclear mRNA export factors (Yi et al., RNA 8:180-7 (2002)). Peptide binding to nucleocytoplasmic transport proteins may thus disrupt tRNA export, mRNA splicing, protein synthesis or microtubule function during the cell cycle.

A fourth set of interactors includes proteins associated with DNA replication, binding, or cell proliferation. A DNA ligase-like protein may join breaks in duplex DNA during replication, repair or recombination. PCNA forms a trimeric ring at the DNA replication fork which is the core of a complex of enzymes involved in DNA replication (Prosperi, E., Prog Cell Cycle Res 3:193-210 (1997)) including a protein critical for replication, DNA polymerase epsilon (Miura, M., J Radiat Res (Tokyo) 40:1-12 (1999)), which was affinity extracted with peptide 35. PCNA is thus a critical protein for DNA replication and cell cycle control (Prosperi, E., Prog Cell Cycle Res 3:193-210 (1997)). Peptide 41 affinity extracts annexin II and calpactin as well as alpha and beta tubulin, actin, myosin, and PCNA. Calpactin is an EF-hand calcium binding protein that forms a cytoskeletal-bound heterotetramer with annexin II (Zokas et al., J Cell Biol 105:2111-21 (1987)). This could explain the presence of tubulin, actin and myosin in the peptide 41 affinity extract. Annexin II is part of the primer recognition complex (Kumble et al, J Cell Sci 101:35-41 (1992); Chiang et al., Mol. Cell. Biochem. 199:139-147 (1999)), and is important for DNA synthesis and cell proliferation. Immunodepletion of annexin II blocks DNA replication (Vishwanatha et al., J. Cell Sci. 105:533-540 (1993)). Peptide 41 could thus block proliferation by interfering with DNA replication. Calpactin and annexin II are involved in calcium signaling (Mueller et al., Histochem Cell Biol 111:453-9 (1999); Hawkins et al., Cell Biochem Biophys 33:275-96 (2000)), as are the endoplasmic reticulum or Golgi-resident multiple EF-hand proteins reticulocalbin, the cytosol and Golgi DNA binding protein nucleobindin/calnuc (Lin et al., Proc Natl Acad Sci USA 97:674-9 (2000)) and calumenin, isolated here by peptide 40.

All three peptides bind importin alpha, importin beta, and ran, and peptide 35 also binds RCC1. Ran regulates microtubule polymerization during mitosis (Wilde et al., Science 284:1359-62 (1999); Kalab et al., Curr Biol 9:481-4 (1999); Kahana et al., J Cell Biol 146:1205-10 (1999)). These four proteins are involved in different ways in spindle assembly during mitosis (Carazo-Salas et al., Nature 400:178-81 (1999); Gruss et al., Cell 12:83-93 (2001); Nachury et al., Cell 104:95-106 (2001)), and the nuclear pore complex in yeast has been reported to functionally interact with components of the spindle assembly checkpoint (Iouk et al., J. Cell. Biol. 159:807-819 (2002)). It is possible that these peptides disregulate this process, resulting in the observed accumulation of cells in M phase by peptides 40 and 41.

The three peptides examined here have nuclear exclusion motifs. These peptides bind a core of similar proteins, have elements of similarity in their sequences, and may block or disregulate a common pathway important to proliferation, such as nucleocytoplasmic transport. Disregulation could result in functional cellular changes in a number of areas, discussed above, that could compromise cell proliferation or cause apoptosis. The three nuclear excluded peptides have a more complex motif than a leucine-rich repeat, which could explain the frequency of these hits of ca. 1 in 10⁸ in the antiproliferative screen. The leucine rich NES motif observed here requires L, I, F, W or M at each of 4 defined positions; this grouping occurs at a frequency of [5/20]⁴. All three peptides have an arg after the second leu of this motif, and a hydrophobic residue before the third leu and after the last leu, which occur at frequencies of 1/20, ˜5/20 and ˜5/20. If the residues before and after the first leu, and after the third leu are required to be R or G, R or L, and R or G respectively, the frequency of occurrence of this entire set of residues is ca. 1/10⁸, similar to the frequency of these peptide hits in the functional screen.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

We claim:

1. A method for identifying a compound that modulates cellular proliferation, the method comprising the steps of:
(i) contacting the compound with a peptide 35, 38, 40, or 41 binding partner; and
(ii) determining the functional effect of the compound upon the binding partner polypeptide.

2. The method of claim 1, wherein the functional effect is measured in vitro.

3. The method of claim 2, wherein the functional effect is a physical effect.

4. The method of claim 3, wherein the functional effect is determined by measuring ligand binding to the binding partner polypeptide.

5. The method of claim 2, wherein the functional effect is a chemical effect.

6. The method of claim 1, wherein the binding partner polypeptide is expressed in a eukaryotic host cell or cell membrane.

7. The method of claim 6, wherein the functional effect is a physical effect.

8. The method of claim 7, wherein the functional effect is determined by measuring ligand binding to the binding partner polypeptide.

9. The method of claim 6, wherein the functional effect is a chemical or phenotypic effect.

10. The method of claim 9, wherein the chemical or phenotypic effect is determined by measuring cellular proliferation.

11. The method of claim 10, wherein the cellular proliferation is measured by assaying for DNA synthesis or fluorescent marker dilution.

12. The method of claim 11, wherein DNA synthesis is measured by ³H thymidine incorporation, BrdU incorporation, or Hoescht staining.

13. The method of claim 11, wherein the fluorescent marker is selected from the group consisting of a cell tracker dye or green fluorescent protein.

14. The method of claim 1, wherein modulation is inhibition of cellular proliferation.

15. The method of claim 1, wherein modulation is inhibition of cancer cell proliferation.

16. The method of claim 6, wherein the host cell is a cancer cell.

17. The method of claim 16, wherein the cancer cell is a breast, prostate, colon, or lung cancer cell.

18. The method of claim 16, wherein the cancer cell is a transformed cell line.

19. The method of claim 18, wherein the transformed cell line is A549.

20. The method of claim 16, wherein the cancer cell is p53 null or mutant.

21. The method of claim 16, wherein the cancer cell is p53 wild-type.

22. The method of claim 1, wherein the polypeptide is recombinant.

23. The method of claim 1, wherein the compound is an antibody.

24. The method of claim 1, wherein the compound is an antisense molecule.

25. The method of claim 1, wherein the compound is a small organic molecule.

26. The method of claim 1, wherein the compound is a peptide.

27. The method of claim 26, wherein the peptide is circular.

28. A method of modulating cellular proliferation in a subject, the method comprising the step of administering to the subject a therapeutically effective amount of a compound identified using the method of claim 1.

29. The method of claim 28, wherein the subject is a human.

30. The method of claim 29, wherein the subject has cancer.

31. The method of claim 28, wherein the compound is an antibody.

32. The method of claim 28, wherein the compound is an antisense molecule.

33. The method of claim 28, wherein the compound is a small organic molecule.

34. The method of claim 28, wherein the compound is a peptide.

35. The method of claim 34, wherein the peptide is circular.

36. The method of claim 28, wherein the compound inhibits cancer cell proliferation.

37. A peptide comprising peptide 35, 38, 40, or 41.
